(19) United States
(12) Patent Application Publication
(10) Pub. No.: US 2026/0071160 A1
(43) Pub. Date: Mar. 12, 2026

(54) SCALABLE BIOREACTOR SYSTEMS AND METHODS FOR TISSUE ENGINEERING

(71) Applicant: FairCraft SAS, Evry-Courcouronnes (FR)

(72) Inventors: Antonio J. PEREIRA-TAVERES, Milpitas, CA (US); George C. ENGELMAYR, Jr., Milpitas, CA (US); Ingvar HELGASON, Milpitas, CA (US)

(21) Appl. No.: 19/387,462

(22) Filed: Nov. 12, 2025

Related U.S. Application Data
(63) Continuation of application No. 17/763,375.
(60) Provisional application No. 62/879,773, filed on Jul. 29, 2019.
(60) Provisional application No. 62/991,958, filed on Mar. 19, 2020.

(51) Int. Cl. C12M 3/00; C12M 1/00; C12M 1/34; C12M 1/36

(52) U.S. Cl. CPC C12M 21/08; C12M 23/48; C12M 23/52; C12M 41/12; C12M 41/48

(57) ABSTRACT

Disclosed herein are scalable, modular bioreactor systems for efficient preparation of cell-based tissues. Also disclosed herein are methods, compositions, and apparatuses for preparing scaffolds.

### CROSS-REFERENCE

This application is a continuation of U.S. patent application Ser. No. 17/763,375, filed Aug. 2, 2022, which is a National Stage Entry of International Patent Application No. PCT/US2020/044123, filed Jul. 29, 2020, which claims the benefit of U.S. Provisional Application No. 62/879,773, filed Jul. 29, 2019, and U.S. Provisional Application No. 62/991,958, filed Mar. 19, 2020, all of which are incorporated herein by reference.

### BACKGROUND

Current tissue engineering bioreactors were neither intended for nor directly applicable in large scale commercial manufacturing. A scalable bioreactor system as described herein can address the need for large scale commercial manufacturing.

### SUMMARY OF THE DISCLOSURE

Disclosed herein are devices. In some embodiments, disclosed herein is a tissue culturing device comprising a plurality of trays substantially within a device, wherein each tray can be configured to hold at least one cell culturing container, wherein trays can be stacked within a device, and wherein a device can be configured to repeatedly tilt such that an angle between a bottom of a stack and a base of a device can repeatedly cycle between about 0 and about 360 degrees. In some embodiments, at least one tray can comprise at least one cell culture container that contains a liquid medium and cells. In some embodiments, at least one tray can be approximately rectangular on a side. In some embodiments, a device can comprise a pivot and a lifting mechanism configured to tilt stacked trays by angular raising and lowering. In some embodiments, an angular raising and lowering can create a wave in a wave in a cell culture medium within said cell culturing container. In some embodiments, a lifting mechanism can comprise a hydraulic mechanism, an electric mechanism, a spring mechanism, a piston, or a combination thereof. In some embodiments, a device can be jacketed. In some embodiments, a device can be temperature controlled. In some embodiments, a base of a device can comprise a pallet. In some embodiments, a device can be stacked on a pallet rack. In some embodiments, a pallet can be detachable. In some embodiments, a pallet can be non-detachable. In some embodiments, a pallet can comprise a plastic, a metal, or a combination thereof. In some embodiments, a pallet can comprise flats, grids, has an elevated foundation such that air can move underneath, or any combination thereof. In some embodiments, a base of a device can comprise means for moving said device using a forklift truck. In some embodiments, a length of a cell culturing container ranges from about 1 cm to about 1000 cm. In some embodiments, a width of a cell culturing container ranges from about 1 cm to about 1000 cm. In some embodiments, a height of a cell culturing container ranges from about 1 cm to about 1000 cm. In some embodiments, a wall thickness of a cell culturing container ranges from about 0.01 cm to about 10 cm. In some embodiments, at least one tray can comprise a handle. In some embodiments, at least one tray can slide in and out of a device. In some embodiments, a device can comprise a monitoring system. In some embodiments, a monitoring system can alert a user that an action must be taken. In some embodiments, a monitoring system comprises, a sensor, a camera, or a combination thereof. In some embodiments, a sensor can comprise a thermistor, a thermometer, a pH sensor, a humidity sensor, a pressure sensor, a smoke detector, or any combination thereof. In some embodiments, a device can be jacketed. In some embodiments, a system can comprise a cell culture medium reservoir. In some embodiments, a cell culture medium reservoir can comprise a monitoring system. In some embodiments, a monitoring system can alert a user that an action must be taken. In some embodiments, a monitoring system comprises, a sensor, a camera, or a combination thereof. In some embodiments, a sensor can comprise a thermistor, a thermometer, a pH sensor, a humidity sensor, a pressure sensor, a smoke detector, or any combination thereof. In some embodiments, a cell culture medium reservoir can be jacketed. In some embodiments, a cell culture medium reservoir can be temperature controlled. In some embodiments, a cell culture medium reservoir can be maintained at a lower temperature than a device. In some embodiments, a cell culture medium can be warmed before entering a device. In some embodiments, a gas can be bubbled into a cell culture medium. In some embodiments, a gas can comprise carbon dioxide, nitrogen, oxygen, or a combination thereof. In some embodiments, a cell culture medium reservoir can be connected to an inlet in a device. In some embodiments, an inlet can comprise an inlet manifold. In some embodiments, a device can comprise an outlet for waste cell culture medium. In some embodiments, an outlet can comprise an outlet manifold. In some embodiments, a system can comprise racks. In some embodiments, racks can be stackable vertically. In some embodiments, racks can be from about 1 m high to about 200 m high. In some embodiments, racks can be arranged from approximately floor to ceiling in a warehouse. In some embodiments, a system can comprise a pallet retrieval system. In some embodiments, a system can be configured to fit on a North American 40″×48″ pallet. In some embodiments, a system can be configured to fit on a conveyer system. In some embodiments, at least one container can comprise a tube operatively connected thereto. In some embodiments, a system can further comprise a temperature control system. A temperature control system can be configured to heat or cool at least a portion of a system to a temperature of from about −200 degrees Celsius to about 200 degrees Celsius. In some embodiments, a system can further comprise an intermediate bulk container heating jacket operatively coupled to a temperature control system. An intermediate bulk container heating jacket can be configured to heat a portion of a system. In some embodiments, a system can further comprise an intermediate bulk container cooling jacket operatively coupled to a temperature control system. An intermediate bulk container cooling jacket can be configured to cool a portion of a system. In some embodiments, a system can further comprise a cell growth substrate. In some embodiments, a cell growth substrate can comprise a synthetic polymer, a natural polymer, a plant-derived material, a microbial-derived material, an animal-derived material, a metal, a ceramic, a glass, a mineral, a rock, a gem, a clay, or any combination thereof. In some embodiments, a cell growth substrate can comprise polystyrene, polyester, polyethylene terephlatate, poly(glycolic acid), poly(lactic acid), poly(lactic-co-glycolic acid), poly(ethylene glycol), polyurethane, poly(glycerol sebacate), polycarbonate, polyetherimide, stainless steel, silver, gold, platinum, palladium, iridium, titanium, tantalum, noble metal, collagen, fibrin, silk, wool, wood, hemp, linin, cotton, cellulose, grass, leaves, straw, lignin, diamond, sapphire, amethyst, ruby, emerald, opal, topaz, quartz, marble, slate, lava rock, coral, sponge, diatom, sand dollar, sea shell, starfish, seaweed, kelp, algae, or any combination thereof. In some embodiments, a cell growth substrate can be positioned inside at least one container. In some embodiments, a cell growth substrate can be positioned using a magnet or a fastener. In some embodiments, at least two containers can be independently rectangular, cylindrical, spherical, or triangular. In some embodiments, at least two containers can be independently constructed from a material that can be substantially rigid, semi-rigid, or flexible. In some embodiments, a motion control system can be an intermediate bulk container tilter. In some embodiments, at least one container can be an intermediate bulk container. In some embodiments, an intermediate bulk container can comprise a volume of at least 275 gallons. In some embodiments, at least one container can be a single use bioprocessing container. In some embodiments, at least one container can be a storage tank. In some embodiments, at least one container can be a pressure vessel. In some embodiments, at least one container can be a drum. In some embodiments, a system can be configured to be heated in an incubator. In some embodiments, a system can be configured to be heated in a heated space. In some embodiments, a system can be configured to be cooled using refrigeration or freezing technology. In some embodiments, at least a portion of at least one container can be gas permeable. In some embodiments, at least a portion of at least one container can be gas impermeable. In some embodiments, at least a portion of an interior of at least one container can be shaped or textured to influence fluid motion. In some embodiments, at least a portion of an interior of at least one container can be shaped or textured to influence cell or tissue attachment. In some embodiments, at least one container can be sterile. In some embodiments, at least one container can be non-sterile. In some embodiments, a device can comprise an intermediate bulk container.

Disclosed herein are systems. A system can be used for growing an animal cell culture. In some cases, a system can comprise at least two containers. In some cases, a system can comprise a motion control system. In some embodiments, at least two containers can be configured for stacking. In some embodiments, an animal cell culture can directly contact a portion of a surface of at least two containers. In some embodiments, a motion control system when activated can be configured to rock or tilt at least two containers at an angle of greater than 0 degrees to about 360 degrees. In some embodiments, at least two stackable containers can be configured to reversibly attach to a motion control system. In some embodiments, a length of at least two containers can range from about 1 cm to about 1000 cm. In some embodiments, a width of at least two containers can range from about 1 cm to about 1000 cm. In some embodiments, a height of at least two containers can range from about 1 cm to about 1000 cm. In some embodiments, a wall thickness of at least two containers can range from about 0.01 cm to about 10 cm. In some embodiments, at least one container can comprise a port operatively coupled to at least one container. In some embodiments, a port can comprise a diameter of from about 0.1 cm to about 1000 cm. In some embodiments, at least one container can comprise a lid. In some embodiments, a lid can be gas permeable. In some embodiments, a lid can be gas impermeable. In some embodiments, a system can further comprise a rack. A rack can comprise a frame and a shelf. In some embodiments, a rack can be configured to reversibly attach to a motion control system. In some embodiments, at least one container can be configured to reversibly attach to a shelf. In some embodiments, a rack, a frame, or a shelf can be independently constructed from a material selected from a group consisting of: polypropylene, polypropylene co-polymers, polyethylene, polyester, polystyrene, polycarbonate, polysulfone, polyolefin, polyetherimide, fluorinated ethylene propylene, polyphenylsulfone, polyetheretherketone, perfluoroalkoxy, ethylene tetrafluoroethylene, ethylene chlorotrifluoroethylene, polychlorotrifluoroethylene, polytetrafluoroethylene, polyphenyl sulfide, silicone, silicone co-polymers, stainless steel, steel alloys, aluminum, aluminum alloys, titanium, titanium alloys, glass, ceramic, and any combination thereof. In some embodiments, a system can be configured to fit on a North American 40″×48″ pallet. In some embodiments, a system can be configured to fit on a conveyer system. In some embodiments, at least one container can comprise a tube operatively connected thereto. In some embodiments, a system can further comprise a temperature control system. A temperature control system can be configured to heat or cool at least a portion of a system to a temperature of from about −200 degrees Celsius to about 200 degrees Celsius. In some embodiments, a system can further comprise an intermediate bulk container heating jacket operatively coupled to a temperature control system. An intermediate bulk container heating jacket can be configured to heat a portion of a system. In some embodiments, a system can further comprise an intermediate bulk container cooling jacket operatively coupled to a temperature control system. An intermediate bulk container cooling jacket can be configured to cool a portion of a system. In some embodiments, a system can further comprise a cell growth substrate. In some embodiments, a cell growth substrate can comprise a synthetic polymer, a natural polymer, a plant-derived material, a microbial-derived material, an animal-derived material, a metal, a ceramic, a glass, a mineral, a rock, a gem, a clay, or any combination thereof. In some embodiments, a cell growth substrate can comprise polystyrene, polyester, polyethylene terephlatate, poly(glycolic acid), poly(lactic acid), poly(lactic-co-glycolic acid), poly(ethylene glycol), polyurethane, poly(glycerol sebacate), polycarbonate, polyetherimide, stainless steel, silver, gold, platinum, palladium, iridium, titanium, tantalum, noble metal, collagen, fibrin, silk, wool, wood, hemp, linin, cotton, cellulose, grass, leaves, straw, lignin, diamond, sapphire, amethyst, ruby, emerald, opal, topaz, quartz, marble, slate, lava rock, coral, sponge, diatom, sand dollar, sea shell, starfish, seaweed, kelp, algae, or any combination thereof. In some embodiments, a cell growth substrate can be positioned inside at least one container. In some embodiments, a cell growth substrate can be positioned using a magnet or a fastener. In some embodiments, at least two containers can be independently rectangular, cylindrical, spherical, or triangular. In some embodiments, at least two containers can be independently constructed from a material that can be substantially rigid, semi-rigid, or flexible. In some embodiments, a motion control system can be an intermediate bulk container tilter. In some embodiments, at least one container can be an intermediate bulk container. In some embodiments, an intermediate bulk container can comprise a volume of at least 275 gallons. In some embodiments, at least one container can be a single use bioprocessing container. In some embodiments, at least one container can be a storage tank. In some embodiments, at least one container can be a pressure vessel. In some embodiments, at least one container can be a drum. In some embodiments, a system can be configured to be heated in an incubator. In some embodiments, a system can be configured to be heated in a heated space. In some embodiments, a system can be configured to be cooled using refrigeration or freezing technology. In some embodiments, at least a portion of at least one container can be gas permeable. In some embodiments, at least a portion of at least one container can be gas impermeable. In some embodiments, at least a portion of an interior of at least one container can be shaped or textured to influence fluid motion. In some embodiments, at least a portion of an interior of at least one container can be shaped or textured to influence cell or tissue attachment. In some embodiments, at least one container can be sterile. In some embodiments, at least one container can be non-sterile.

Also disclosed herein are methods of growing an animal cell culture. A method can comprise culturing a cell culture in a system described herein. In some embodiments, a culturing can produce a cell-based tissue. In some embodiments, a cell-based tissue can be tanned, retanned, dyed, or fatliquored. In some embodiments, an animal cell culture can comprise a transgene, a heterologous RNA, or an epigenetically modified base.

Also disclosed herein are cell-based tissues produced by a method described herein. In some embodiments, a cell-based tissue can be treated for a purpose of making leather.

Also disclosed herein are compositions. In some cases, a composition can comprise an acrylated chitosan. In some cases, a composition can comprise an acrylate polymer. In some cases, an acrylate polymer can comprise a phosphine oxide or a salt thereof. In some cases, an acrylated chitosan can be a methacrylated chitosan. In some cases, an acrylated chitosan can be a methacrylated chitosan salt. In some cases, an acrylated polymer can comprise polyethylene glycol. In some cases, an acrylated polymer can be poly(ethylene glycol) dimethacrylate. In some cases, an acrylated polymer can comprise polyethylene glycol. In some cases, an acrylated polymer can be a poly(ethylene glycol) dimethacrylate salt. In some cases, a phosphine oxide can be 2,4,6-trimethylbenzoyl-diphenylphosphine oxide. In some cases, a phosphine oxide can be a 2,4,6-trimethylbenzoyl-diphenylphosphine oxide salt. In some cases, a salt can comprise a lithium salt. In some cases, a salt can comprise a sodium salt. In some cases, a composition can further comprise an organic dye. In some cases, a composition can further comprise an organic dye salt. In some cases, an organic dye or salt thereof can be Martius yellow or a salt thereof. In some cases, a composition can comprise a sodium salt of Martius yellow.

Also disclosed herein are methods that can comprise contacting an acrylated chitosan with an acrylate polymer. In some cases, an acrylate polymer can comprise a phosphine oxide. In some cases, an acrylate polymer can comprise a phosphine oxide salt. In some cases, an acrylated chitosan can be a methacrylated chitosan. In some cases, an acrylated chitosan can be a methacrylated chitosan salt. In some cases, an acrylated polymer can comprise polyethylene glycol. In some cases, an acrylated polymer can be poly(ethylene glycol) dimethacrylate. In some cases, an acrylated polymer can be a poly(ethylene glycol) dimethacrylate salt. In some cases, a phosphine oxide or salt thereof can be 2,4,6-trimethylbenzoyl-diphenylphosphine oxide or a salt thereof. In some cases, a phosphine oxide can be a salt of a 2,4,6-trimethylbenzoyl-diphenylphosphine oxide. In some cases, a phosphine oxide can comprise a lithium salt of a 2,4,6-trimethylbenzoyl-diphenylphosphine oxide. In some cases, a phosphine oxide can comprise a sodium salt of a 2,4,6-trimethylbenzoyl-diphenylphosphine oxide. In some cases, a method can further comprise contacting an acrylated chitosan with an organic dye or a salt thereof. In some cases, a method can further comprise contacting an acrylate polymer with an organic dye or a salt thereof. In some cases, an organic dye or salt thereof can be Martius yellow or a salt thereof. In some cases, an organic dye can be a sodium salt of Martius yellow. In some cases, an acrylated chitosan can be dissolved in an organic acid prior to contacting with an acrylate polymer. In some cases, a method can further comprise printing a scaffold. In some cases, a scaffold can comprise an acrylated chitosan. In some cases, a scaffold can comprise an acrylate polymer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A depicts a substantially rectangular container with a length (l), width (w), height (h), and wall thickness (t), where each container has a plurality of at least one openings or ports and a plurality of at least one tops or lids.

FIG. 1B depicts a substantially cylindrical container.

FIG. 1C depicts a substantially spherical container.

FIG. 2A depicts a schematic of a production module (i.e., tissue-engineering bioreactor system).

FIG. 2B depicts a production module.

FIG. 3 depicts a schematic of a palletized tissue-engineering bioreactor system.

FIG. 4 depicts a schematic rear view of an exemplary embodiment of a palletized tissue-engineering bioreactor system. Fluid inlet and outlet manifolds are indicated, where a plurality of tubing or piping is depicted supplying and removing fluid to a plurality of containers via a plurality of openings or ports.

FIG. 5 depicts a schematic side view of an exemplary embodiment of a palletized tissue-engineering bioreactor system.

FIG. 6 depicts an image of a warehouse pallet racking system with automated pallet storage and retrieval.

FIG. 7A depicts an example image of a plastic pallet for storage and shipping. FIG. 7B depicts an example image of an aluminum pallet for storage and shipping. FIG. 7C depicts an example image of a stainless-steel pallet for storage and shipping. Standard pallets in North America are 48″×40″×6″ Standard pallets in North America are 48″×40″×6″.

FIG. 8A and FIG. 8B depicts example images of small pallet rack and larger pallet racks in a typical warehouse.

FIG. 9A depicts example images of Intermediate Bulk Containers (IBC) with integrated pallet for fluid containment and transport (275 gallon (˜1000 liter)). FIG. 9B depicts example images of Intermediate Bulk Containers (IBCs) with integrated pallet and heating jackets. FIG. 9C depicts example images of Intermediate Bulk Containers (IBCs) with integrated pallet and cooling jacket and chiller.

FIG. 10A depicts an image of Intermediate Bulk Containers (IBCs) stacked on a pallet rack in a warehouse. FIG. 10B depicts an example image of empty pallet racks in warehouse.

FIG. 11A, FIG. 11B, FIG. 11C, FIG. 11D, and FIG. 11E depict images of IBC tilters which can be used to ensure complete drainage of liquid from an IBC.

FIG. 12 depicts collagen concentration measured biochemically at five distinct locations in an exemplary cell-based bovine skin tissue grown via an embodiment of a system disclosed herein.

FIG. 13 depicts an example of a cell growth substrate.

FIG. 14 depicts an outline of the process of manufacturing a synthetic leather.

### DETAILED DESCRIPTION OF THE DISCLOSURE

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

In this disclosure the term “about” or “approximately” can mean a range of up to 10% of a given value. In this disclosure the term “substantially” refers to something that is done to a great extent or degree.

As used herein, the term “pluripotent stem cell” can refer to any precursor cell that has the ability to form any adult cell.

As used herein, the term “embryonic stem cells” or “ES cells” or “ESC” can refer to precursor cells that have the ability to form any adult cell.

As used herein, the term “induced pluripotent stem cells” or “iPS cells” or “iPSCs” can refer to a type of pluripotent stem cell artificially derived from a non-pluripotent cell (e.g., an adult somatic cell). Induced pluripotent stem cells can be identical to embryonic stem cells in the ability to form any adult cell but are not derived from an embryo.

As used herein, the term “synthetic leather” can refer to leather made from cultured cells, for example, skin equivalents. Skin equivalents described herein can serve as a skin equivalent for any mammal or non-mammal. A skin equivalent can be for human and non-human mammals, such as non-human primates and members of the bovine, ovine, porcine, equinine, canine and feline species as well as rodents such as mice, rats and guinea pigs, members of the lagomorph family including rabbit, fish including shark and stingray, birds including ostrich and reptiles including lizards, snakes and crocodiles. The particular mammalian synthetic leather which will be formed can be dependent on the source of the cells as described herein, e.g. Keratinocytes and fibroblasts, e.g., when bovine keratinocytes and fibroblasts are used to form a skin equivalent, a bovine synthetic leather can be formed.

The term “chitosan”, as used herein, will be understood by those skilled in the art to include all derivatives of chitin, or poly-N-aceryl-D-glucosamine (including all polyglucosamine and oligomers of glucosamine materials of different molecular weights), in which the greater proportion of the N-acetyl groups have been removed through hydrolysis. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit). Chitosan can have a positive charge. Chitosan, chitosan derivatives or salts (e.g., nitrate, phosphate, sulphate, hydrochloride, glutamate, lactate or acetate salts) of chitosan may be used and are included within the meaning of the term “chitosin”. As used herein, the term “chitosan derivatives” are intended to include ester, ether or other derivatives formed by bonding of acyl and/or alkyl groups with OH groups, but not the NH₂ groups, of chitosan. Examples are O-alkyl ethers of chitosan and O-acyl esters of chitosan. Modified chitosans, particularly those conjugated to polyethylene glycol, are included in this definition. Low and medium viscosity chitosans (for example CL113, G210 and CL110) may be obtained from various sources, including PRONOVA Biopolymer, Ltd. (UK); SEIGAGAKU America Inc. (Maryland, USA); MERON (India) Pvt, Ltd. (India); VANSON Ltd. (Virginia, USA); and AMS Biotechnology Ltd. (UK). Suitable derivatives include those which are disclosed in Roberts, Chitin Chemistry, MacMillan Press Ltd., London (1992). Optimization of structural variables such as the charge density and molecular weight of the chitosan is contemplated and encompassed by the present disclosure.

A “chitosan” (or chitosan derivative or salt) can have a molecular weight of 1,000 Dalton (Da) or more, for example in the range 1,000 to 4,000, 4,000 to 10,000, 10,000 to 20,000, 20,000 to 50,000, 50,000 to 100,000, 100,000 to 150,000, or 150,000 to 300,000. In some cases, chitosan or chitosan powder can have a molecular weight of 150,000 Daltons. Chitosans of different low molecular weights can be prepared by enzymatic degradation of chitosan using chitosanase or by the addition of nitrous acid. In some embodiments, the chitosan can be water-soluble and may be produced from chitin by deacetylation to a degree of greater than 40%, between 50% and 98%, or between 70% and 90%.

The term “PEGDA” can refer to poly(ethylene glycol) dimethacrylate. In some aspects, a PEGDA resin can comprise Li-TPO and/or Martius yellow. In some aspects, a PEGDA resin can comprise between 0.01 and 5% Li-TPO. In some embodiments, a PEGDA resin can comprise at least about 0.01, 0.02, 0.03, 0.04, 0.05, 0.06, 0.07, 0.07, 0.08, 0.09, 0.1, 0.2, 0.3, 0.4, 0.5, 0.6, 0.7, 0.8, 0.9 1.0, 1.5, 2.0, 3.0, 4.0, or at least about 5% Li-TPO. In some aspects, a PEGDA resin can comprise between 0.001 and 5% Martius yellow. In some embodiments, a PEGDA resin can comprise at least about 0.001, 0.005, 0.009, 0.01, 0.02, 0.03, 0.04, 0.05, 0.06, 0.07, 0.07, 0.08, 0.09, 0.1, 0.2, 0.3, 0.4, 0.5, 0.6, 0.7, 0.8, 0.9 1.0, 1.5, 2.0, 3.0, 4.0, or at least about 5% Martius yellow. PEGDA can have a molecular weight of 100 Dalton or more, for example in the range 100 to 500, 500 to 1,000, 1,000 to 4,000, 4,000 to 10,000, 10,000 to 20,000, 20,000 to 50,000, 50,000 to 100,000, 100,000 to 150,000, or 150,000 to 300,000. In some embodiments, PEGDA can have a molecular weight of 700 Daltons.

The term “biocompatible” can refers to the absence of stimulation of a severe, long-lived or escalating biological response to a product or coating, and is distinguished from a mild, transient inflammation which typically accompanies surgery or implantation of foreign objects into a living organism.

The terms “biodegradable” and “bioerodible” can refer to the dissolution of an implant or coating into constituent parts that may be metabolized or excreted, under the conditions normally present in a living tissue. In exemplary embodiments, the rate and/or extent of biodegradation or bioerosion may be controlled in a predictable manner.

The term “co-depositing” can describe the placement of two or more substances, at the same position in, for example, a scaffold. Substances may be co-deposited simultaneously or non-simultaneously (for example, sequentially).

“Bioreactor” can refer to any device or system that supports a biologically active environment, for example for cultivation of cells or organisms for production of a biological product. This would include cell stacks, roller bottles, shakes, flasks, stirred tank suspension bioreactors, high cell density fixed bed perfusion bioreactors, etc.

### Overview

Disclosed herein are scalable, modular systems for the production of cell culture, for example, animal cell culture. A system disclosed herein can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 containers. In some cases, the containers can be configured for stacking onto each other or shelves and are capable of growing cultured cells and tissue. In some cases, when placed in a system as described herein, an animal cell culture can directly contact a portion of an interior surface of a container of the system. The system may further comprise a motion control system, wherein the motion control system can be configured to rock or tilt a container at an angle of greater than 0 degrees to about 360 degrees when activated. To allow for removability of containers, in some cases a container disclosed herein can be configured to be reversibly attached to another container, a shelve or the motion control system. In some embodiments, reversible attachment can comprise a reversible locking of a container to a motion control system. In some embodiments, reversible attachment can comprise a physical connection of a container to a motion control system. In some embodiments, reversible attachment can comprise a container being held in place by a motion control system. In some embodiments, a container held in place by a motion control system can only substantially move around one axis. In some embodiments, a container held in place by a motion control system can only substantially move in one plane. Further disclosed herein are methods of growing cell culture, e.g. animal cell culture, in a system as described herein.

Also disclosed herein are compositions and methods for developing scaffolds for use in tissue engineering, for example, engineered dermal equivalent, engineered epidermal equivalent, or engineered full thickness skin equivalent. In some cases, a scaffold described herein can be a biocompatible scaffold. A biocompatible scaffold can be made of natural material(s) of non-animal origin alone or with supplements. In some aspects, a scaffold described herein can comprise supplements or agents for modification of physical characteristics of the scaffold (e.g. elasticity, flexibility etc.), and/or for 3D printing process of specific patterns. In some cases, cells or tissues can be grown/cultured in a system disclosed herein on a scaffold disclosed herein.

A scaffold disclosed herein can possess at least one of the following characteristics for use in tissue engineering: (i) a three-dimensional porous structure that allows cell/tissue growth maintaining or pursuing desired cellular phenotype and flow transport of nutrients and metabolic waste; (ii) biodegradable or bioresorbable with a controllable degradation and resorption rate to match cell/tissue growth in vitro and/or in vivo with a timely or final complete removal or clearance; (iii) conducive surface chemistry for cell attachment, proliferation, and differentiation; (iv) mechanical properties to match natural tissues; and (v) processability to form a variety of shapes and sizes for various applications. Further disclosed herein are methods of printing a scaffold disclosed herein using a 3-D printing technique.

### Bioreactors

Early tissue engineering bioreactors were neither intended for nor directly applicable in large-scale commercial manufacturing. Further, many such bioreactors are static and limited by the scale of the container wherein which cells are grown and often cannot utilize additional containers when scaling up production.

In contrast, disclosed herein is a scalable, stackable, modular system for growing cells, e.g. animal cells or tissue. Further, in some embodiment, the system disclosure herein can be configured to allow easy, rapid scaling up. Furthermore, a system described herein can include a motion control system configured to rock or tilt a container, which can create a dynamic flow within the chamber that can increase viability of cell culture within the container. In some embodiments, a bioreactor can include a container in which cells are grown, a temperature regulation system, a gas inlet/outlet system configured to regulate a gas concentration within the container, an agitator configured to mix growth medium within a container, any number of inlet/outlet ports for fluid transport. In some embodiments, a cell culture can be suspended in a growth medium in the container of the bioreactor. In some embodiments, a user can program settings that can dictate a speed of agitation, a desired pH, a temperature, and or a dissolved oxygen level. In some embodiments, a system can adjust these parameters based on the parameters input by the user. In some embodiments, a user or this system can manually or automatically siphon/change media or other components using, for example, a pump operatively connected to a port in a container via a tube or pipe. In some embodiments, media can continuously flow in and out of a container of a system. In some embodiments, the system can utilize static motion (i.e. the bioreactor typically remains stationary during the growth). In some embodiments, a system described herein can include a motion control system configured to rock, rotate, or tilt a container, which can create a dynamic flow within the chamber that can increase viability of cell culture within the container.

### Medium

In some embodiments, any growth medium can be used to grow a cell culture in a system described herein. Non-limiting examples includes but are not limited to Eagle's minimal essential medium (MEM), Dulbecco's modified Eagle's medium (DMEM), Iscove basic DMEM, Roswell Park Memorial Institute medium (RPMI), 199/109 media, HamF10/HamF12 media, McCoy's 5A medium, or any combination thereof. In some embodiments, a medium can comprise a differentiation agent as disclosed herein. In some embodiments, a differentiation agent can comprise a small molecule, a growth factor, a hormone, a serum, or any combination thereof.

In some embodiments, cells can be expanded in DMEM with 10% fetal bovine serum. In some embodiments, a tissue formation can occur in DMEM with human platelet lysate, ascorbate, TGFβ, or any combination thereof. In some embodiments, a medium formulation can be substantially devoid of animal components. In some embodiments, a medium can be xeno-free. In some embodiments, a medium can support cell adhesion and tissue formation. In some embodiments, production of a medium can be scaled to millions of liters per year. In some embodiments, a medium can be carbon dioxide independent. In some embodiments, a medium can be substantially free of carbon dioxide. In some embodiments, a medium can contain small molecules to stimulate specific pathways for stimulation of matrix protein production. In some embodiments, a medium can contain growth factors.

### Container

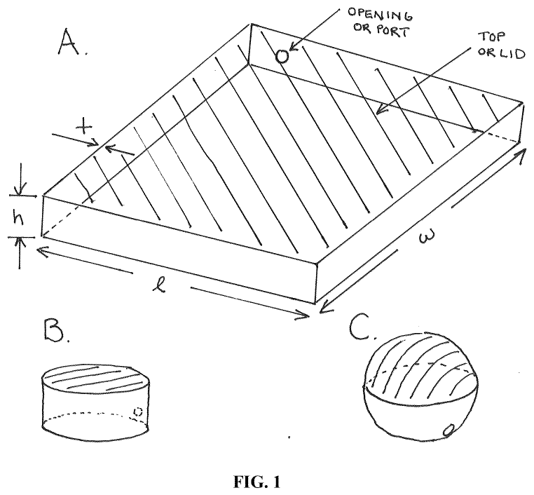

FIG. 1A. depicts one exemplary embodiment of a substantially rectangular container having a length, width, and height. Materials of construction of a container (an example of which is polypropylene) can have a thickness. In some cases, the purpose of a container can be to contain any combination of a cell-growth substrate, cell, fluid media, a gas (e.g., oxygen, nitrogen, carbon dioxide, argon, carbon monoxide, air), frame, mechanical stimulation equipment, baffles, flow directing elements, cell-based tissue, and/or a product resulting from or a derivative of a cell or a tissue.

While FIG. 1A. depicts a substantially rectangular container, a container can have any number of shapes. For example, a container may be configured as a sphere, cone, pyramid, cube, cylinder, prism, tetrahedron, cuboid, octahedron, dodecahedron, ellipsoid, icosahedron, and the like. Further, a surface of a container can be textured, grooved, or otherwise shaped or textured to allow for increased fluid turbulence, or to facilitate cellular attachment, during the cell culturing. For example, a container can contain a grooved surface on a bottom surface that allows a cell culture to anneal into or on a groove. A groove can be created, for example, using etching or carving into the surface of the container. In some embodiments, an internal or external surface of a container can be configured, patterned or textured specific to a purpose of a culture being grown. Further, a container can contain features such as baffles, points, or other raised surfaces that enhance fluid turbulence. Such features can be prepared by fabrication, or by annealing or fastening objects to, into or on the container. A container can also have both a raised surface and a grooved surface to provide both features.

In some embodiments, cells to be cultured can be directly added to a container, such that a cell culture can directly contact a portion of a surface of a container. In some cases, a cell culture or growth medium can be placed in a membrane or bag in a container. In some cases, a membrane or bag are not utilized. In some cases, a cell culture/tissue culture can be grown such that the cell culture does not contact an internal surface of a container. In some embodiments, tissue culture and cell culture can be used interchangeably.

In some embodiments, a container can have a length ranging from at least about 1 cm to at least about 1000 cm, a width ranging from about 1 cm to about 1000 cm, a height ranging from about 1 cm to about 1000 cm, or a thickness ranging from about 0.01 cm to about 10 cm. In some embodiments, a container can have a length of from about 1 cm to about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm.

In some embodiments, a container can have a width of from at least about 1 cm to at least about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm.

In some embodiments, a container can have a height of from at least about 1 cm to at least about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm.

In some embodiments, a container can have a thickness of from at least about 0.01 cm to at least about 10 cm, from about 0.01 cm to about 9 cm, from about 0.01 cm to about 8 cm, from about 0.01 cm to about 7 cm, from about 0.01 cm to about 6 cm, from about 0.01 cm to about 5 cm, from about 0.01 cm to about 4 cm, from about 0.01 cm to about 3 cm, from about 0.01 cm to about 2 cm, from about 0.01 cm to about 1 cm, from about 0.01 cm to about 0.9 cm, from about 0.01 cm to about 0.8 cm, from about 0.01 cm to about 0.7 cm, from about 0.01 cm to about 0.6 cm, from about 0.01 cm to about 0.5 cm, from about 0.01 cm to about 0.4 cm, from about 0.01 cm to about 0.3 cm, from about 0.01 cm to about 0.2 cm, or from about 0.01 cm to about 0.1 cm.

In some embodiments, a substantially rectangular container can have a length ranging from at least about 35 inches (i.e., about 89 cm) to at least about 50 inches (i.e. about 127 cm), a width ranging from at least about 35 inches (i.e., about 89 cm) to at least about 50 inches (i.e. 127 cm), a height ranging from at least about 0.25 inches (i.e., about 0.6 cm) to at least about 5 inches (i.e., about 12.7 cm), and a material of construction thickness ranging from at least about 0.1 inches (i.e., about 0.25 cm) to at least about 0.5 inches (i.e., about 1.3 cm). In some embodiments, a substantially rectangular container can have a length of at least about 38 inches (i.e., about 96.5 cm), a width of at least about 38 inches (i.e., about 96.5 cm), a height of at least about 1.5 inches (i.e., about 3.8 cm), and a material of construction thickness of at least about 3/16 inches (i.e., about 0.1875 inches, or about 0.476 cm). In some embodiments, the top, bottom, and four sides of one or more of the substantially rectangular containers may be fabricated from the same materials of construction or from different materials of construction.

In some cases, a container can be a standardized industrial container used for storing or transporting bulk liquids or powders. For example, a container can be an intermediate bulk container (IBC) (i.e. IBC tote, IBC tank, or pallet tank). Such reusable industrial-grade containers can be used in a system described herein as containers for cell culture. In some cases, a standardized 275 gallon or a 330-gallon IBC can be used. Other industrial containers that can be used include a storage tank, a pressure vessel, a drum, a jug, and the like.

Referring to FIG. 1A, in some exemplary embodiments, a container can include at least one, two, three, four, five, six, seven, eight, ten, or fifteen openings or ports. An opening or port can have a diameter, or equivalent dimensions of an opening or port, from about 0.1 cm to about 1000 cm, from about 0.1 cm to about 900 cm, from about 0.1 cm to about 800 cm, from about 0.1 cm to about 700 cm, from about 0.1 cm to about 600 cm, from about 0.1 cm to about 500 cm, from about 0.1 cm to about 400 cm, from about 0.1 cm to about 300 cm, from about 0.1 cm to about 200 cm, from about 0.1 cm to about 100 cm, from about 0.1 cm to about 90 cm, from about 0.1 cm to about 80 cm, from about 0.1 cm to about 70 cm, from about 0.1 cm to about 60 cm, from about 0.1 cm to about 50 cm, from about 0.1 cm to about 40 cm, from about 0.1 cm to about 30 cm, from about 0.1 cm to about 20 cm, from about 0.1 cm to about 10 cm, from about 0.1 cm to about 9 cm, from about 0.1 cm to about 8 cm, from about 0.1 cm to about 7 cm, from about 0.1 cm to about 6 cm, from about 0.1 cm to about 5 cm, from about 0.1 cm to about 4 cm, from about 0.1 cm to about 3 cm, from about 0.1 cm to about 2 cm, or from about 0.1 cm to about 1 cm. In one embodiment, a container can include one opening or port, wherein the diameter or equivalent dimensions of an opening or port ranges from about 0.3 cm to about 1.3 cm.

FIG. 1A depicts one embodiment, in which an opening or port can be located on one of four sides of a substantially rectangular container at a distance from any side edge (i.e., side corner) of the container to the center of the opening or port. For example, an opening or port may be located at a distance of from about 0.5 cm to about 1000 cm, from about 0.5 cm to about 900 cm, from about 0.5 cm to about 800 cm, from about 0.5 cm to about 700 cm, from about 0.5 cm to about 600 cm, from about 0.5 cm to about 500 cm, from about 0.5 cm to about 400 cm, from about 0.5 cm to about 300 cm, from about 0.5 cm to about 200 cm, from about 0.5 cm to about 100 cm, from about 0.5 cm to about 95 cm, from about 0.5 cm to about 90 cm, from about 0.5 cm to about 85 cm, from about 0.5 cm to about 80 cm, from about 0.5 cm to about 75 cm, from about 0.5 cm to about 70 cm, from about 0.5 cm to about 65 cm, from about 0.5 cm to about 60 cm, from about 0.5 cm to about 55 cm, from about 0.5 cm to about 50 cm, from about 0.5 cm to about 45 cm, from about 0.5 cm to about 40 cm, from about 0.5 cm to about 35 cm, from about 0.5 cm to about 30 cm, from about 0.5 cm to about 25 cm, from about 0.5 cm to about 20 cm, from about 0.5 cm to about 15 cm, from about 0.5 cm to about 10 cm, from about 0.5 cm to about 9 cm, from about 0.5 cm to about 8 cm, from about 0.5 cm to about 7 cm, from about 0.5 cm to about 6 cm, or from about 0.5 cm to about 5 cm from any side edge. An opening or port may also be located at a distance ranging from about 0.5 cm to about 100 cm, from about 0.5 cm to about 95 cm, from about 0.5 cm to about 90 cm, from about 0.5 cm to about 85 cm, from about 0.5 cm to about 80 cm, from about 0.5 cm to about 75 cm, from about 0.5 cm to about 70 cm, from about 0.5 cm to about 65 cm, from about 0.5 cm to about 60 cm, from about 0.5 cm to about 55 cm, from about 0.5 cm to about 50 cm, from about 0.5 cm to about 45 cm, from about 0.5 cm to about 40 cm, from about 0.5 cm to about 35 cm, from about 0.5 cm to about 30 cm, from about 0.5 cm to about 25 cm, from about 0.5 cm to about 20 cm, from about 0.5 cm to about 15 cm, from about 0.5 cm to about 10 cm, from about 0.5 cm to about 9 cm, from about 0.5 cm to about 8 cm, from about 0.5 cm to about 7 cm, from about 0.5 cm to about 6 cm, or from about 0.5 cm to about 5 cm from any bottom edge (i.e., bottom corner) of the container to the center of said opening or port.

In an exemplary embodiment, a container can include an opening or port, wherein the diameter of the opening or port can be about 0.25 inches (i.e., about 0.635 cm) and where the opening or port can be located on one of the four sides of the container at a distance of about 19 inches (i.e., about 48.26 cm) from any side edge (i.e., side corner) of the container to the center of the opening or port and at a distance of about 0.3125 inches (i.e., about 0.79 cm) from any bottom edge (i.e., bottom corner) of the container to the center of the opening or port. In an exemplary embodiment, a container can include two openings or ports, where a diameter or equivalent dimensions of the openings or ports ranges from about 0.3 cm to about 1.3 cm, and where the openings or ports can be located on one of the four sides of the container at a distance ranging from about 0.5 cm to about 65 cm from any side edge (i.e., side corner) of the container to the center of the openings or ports and at a distance ranging from about 0.5 cm to about 10 cm from any bottom edge (i.e., bottom corner) of the container to the center of said openings or ports. In an exemplary embodiment, a container can include two openings or ports, wherein a diameter of the openings or ports can be about 0.25 inches (i.e., about 0.635 cm) and where the openings or ports are located on one of the four sides of the container at a distance of about 3 inches (i.e., about 7.62 cm) from any side edge (i.e., side corner) of the container to the center of said openings or ports, and at a distance of about 0.3125 inches (i.e., about 0.79 cm) from any bottom edge (i.e., bottom corner) of the container to the center of the openings or ports.

In some embodiments, an opening or port may be circular. In some embodiments, an opening or port may not be circular, but rather can have any shape, including but not limited to oval, square, elliptical, or rectangular. In some embodiments, the openings or ports may be on the sides of the container, the bottom of the container, the top or lid of the container, or in any combination of locations and numbers. A container may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 openings or ports. In some embodiments, an opening or port may be a simple hole formed by drill, laser, machining, molding, extrusion, casting, or any other additive or subtractive manufacturing method. In some embodiments, an opening or port may be a fitting, including but not limited to a barbed fitting, a flanged fitting, a sanitary fitting, etc. In some embodiments, an opening or port may include a valve. In some embodiments, a value can be a one-way valve or a two way valve. In some embodiments, an opening or port may include a cap. In some embodiments a cap can be a solid cap or a cap having an opening. In some embodiments, a cap opening can comprise a filter. In some embodiments, a port or opening can comprise a filter.

A container may include at least one top or lid. A top or a lid may have a length of from about 1 cm to about 1100 cm, from about 1 cm to about 1000 cm, from about 1 cm to about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm. A top or a lid may have a width of from about 1 cm to about 1100 cm, from about 1 cm to about 1000 cm, from about 1 cm to about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm. A top or a lid may have a height of from about 0.005 to about 100 cm, from about 0.005 to about 90 cm, from about 0.005 to about 80 cm, from about 0.005 to about 70 cm, from about 0.005 to about 60 cm, from about 0.005 to about 50 cm, from about 0.005 to about 40 cm, from about 0.005 to about 30 cm, from about 0.005 to about 20 cm, from about 0.005 to about 10 cm, from about 0.005 to about 9 cm, from about 0.005 to about 8 cm, from about 0.005 to about 7 cm, from about 0.005 to about 6 cm, from about 0.005 to about 5 cm, from about 0.005 to about 4 cm, from about 0.005 to about 3 cm, from about 0.005 to about 2 cm, from about 0.005 to about 1 cm, from about 0.005 to about 0.9 cm, from about 0.005 to about 0.8 cm, from about 0.005 to about 0.7 cm, from about 0.005 to about 0.6 cm, from about 0.005 to about 0.5 cm, from about 0.005 to about 0.4 cm, from about 0.005 to about 0.3 cm, from about 0.005 to about 0.2 cm, or from about 0.005 to about 0.1 cm. A top or a lid may have a wall thickness of from about 0.005 to about 50 cm, from about 0.005 to about 40 cm, from about 0.005 to about 30 cm, from about 0.005 to about 20 cm, from about 0.005 to about 10 cm, from about 0.005 to about 9 cm, from about 0.005 to about 8 cm, from about 0.005 to about 7 cm, from about 0.005 to about 6 cm, from about 0.005 to about 5 cm, from about 0.005 to about 4 cm, from about 0.005 to about 3 cm, from about 0.005 to about 2 cm, from about 0.005 to about 1 cm, from about 0.005 to about 0.9 cm, from about 0.005 to about 0.8 cm, from about 0.005 to about 0.7 cm, from about 0.005 to about 0.6 cm, from about 0.005 to about 0.5 cm, from about 0.005 to about 0.4 cm, from about 0.005 to about 0.3 cm, from about 0.005 to about 0.2 cm, or from about 0.005 to about 0.1 cm.

In an exemplary embodiment, a top or lid can have a length ranging from at least about 35 inches (i.e., about 89 cm) to at least about 51 inches (i.e. about 129.5 cm), a width ranging from at least about 35 inches (i.e., about 89 cm) to at least about 51 inches (i.e. 129.5 cm), a height ranging from at least about 0.002 inches (i.e., about 0.005 cm) to at least about 4 inches (i.e., about 10.16 cm), and a material of construction thickness ranging from at least about 0.1 inches (i.e., about 0.25 cm) to at least about 0.5 inches (i.e., about 1.3 cm). In an exemplary embodiment, a top or lid can have a length of about 38.625″ (i.e., about 98.1 cm), width of about 38.625″ (i.e., about 98.1 cm), height of about 1.1875 inches (i.e., about 3.016 cm), and material of construction thickness of about 3/16 inches (i.e., about 0.1875 inches, or about 0.476 cm).

In some embodiments, a container as described herein can be configured to be stackable. The term “stackable” can refer to a property in which a plurality of containers can be stacked on top of each other. In some cases, a container may be directly placed on top a second container. In some cases, a container may be stacked on top of another while separated by a shelf or rack. In some cases, a stackable container may include a plurality of containers stacked by means of a slidable shelf. An individual can slide out or into a system while in a stacked configuration through the use of a slidable rack.

At least two containers can be stacked onto each other and operatively connected to a system as described herein. While various shapes of containers are described herein, where a plurality of containers are to be stacked, a compatible shape can be chosen such that a plurality of containers are capable of being stacked. In some cases, a container configured to be stacked can comprise a fitting or an adapter configured to facilitate stacking. For example, a container may comprise a grooved recess on a top or a bottom surface or lid, which can be designed to interlock onto a second container. Further, a pair of adapters having a male end and a female end can be employed. In some cases, two or more containers can be fastened or joined together. Examples of attachments that can be used to fasten or join components of the apparatus to one another can include a glue, an epoxy, a hook- and loop fastener (e.g. Velcro®), a weld, a magnet, a screw, a ball bearing, a staple, a rivet, and any combination thereof. In some instances, the modular components of the apparatus can comprise low coefficients of friction at an attachment surface. In such a configuration, friction between the two surfaces can serve to fasten or join the components together.

A system as described herein can include any number of containers for producing a cell culture as described herein, including operation of a single container. Where a stacked configuration is employed, a plurality of containers can be use. In some embodiments, a system can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, or at least 200 containers.

In some embodiments, the top, bottom, or a side of tops or lids of a container may be fabricated from the same materials of construction or from different materials of construction, both from the top or lid components and/or from the container components.

In some embodiments, at least a portion of a lid can be constructed from an at least partially gas permeable material, such as silicone, fluorinated ethylene propylene, Tyvek, or the like. In some embodiments, the material of construction can be chosen to maximize gas permeability (e.g., to enable oxygen and carbon dioxide exchange) while minimizing water vapor permeability (e.g., to prevent evaporative losses from the container). In some embodiments, a lid can incorporate a gasket and a lid closure, such as screws, clamps, latches, and the like. In some embodiments, a lid can fit relatively loosely or relatively tightly around the top edges of the sides of the container, such as in the lid of a standard Petri dish.

Referring to FIG. 1B and FIG. 1C, a container may be cylindrical, spherical, or any other more complex shape, including but not limited to those formed by a combination of shapes and triangular shapes. Accordingly, a top or lid may be cylindrical, spherical, or any other more complex shape, including but not limited to those formed by a combination of shapes and triangular shapes. In some embodiments, a container disclosed herein can be sterilized.

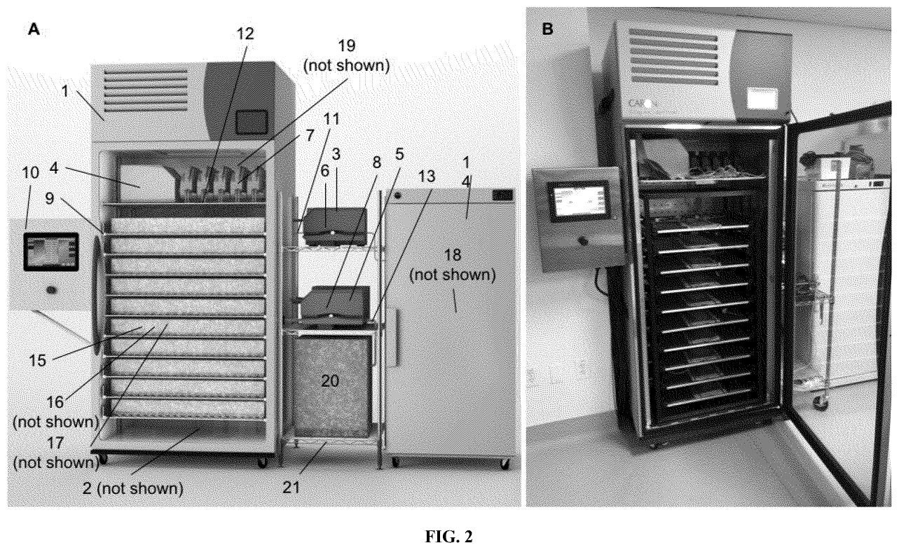

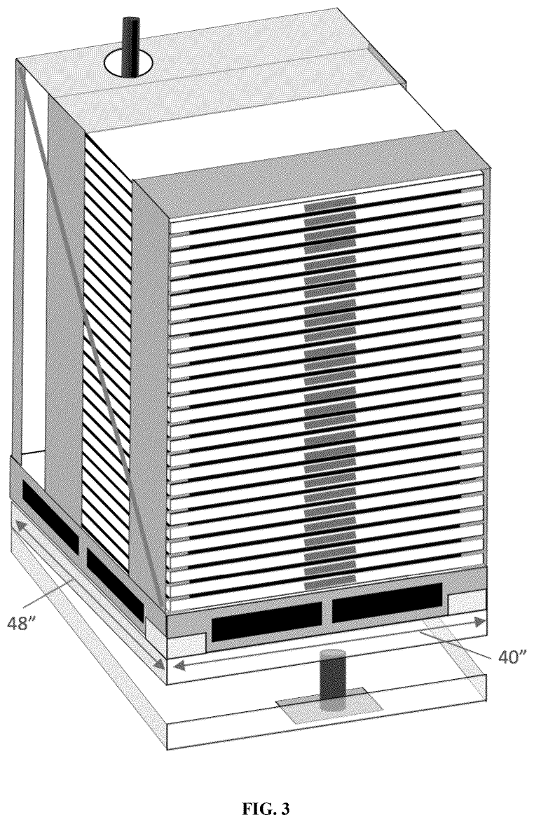

Referring to FIG. 2 and FIG. 3, a system can comprise a rack comprising a frame and at least one shelf configured to support a container as described herein. In some embodiments, a shelf can have a length of from at least about 1 cm to at least about 2000 cm, from about 1 cm to about 1900 cm, from about 1 cm to about 1800 cm, from about 1 cm to about 1700 cm, from about 1 cm to about 1600 cm, from about 1 cm to about 1500 cm, from about 1 cm to about 1400 cm, from about 1 cm to about 1300 cm, from about 1 cm to about 1200 cm, from about 1 cm to about 1100 cm, from about 1 cm to about 1000 cm, from about 1 cm to about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm. In some embodiments, a shelf can have a width of from about 1 cm to about 2000 cm, from about 1 cm to about 1900 cm, from about 1 cm to about 1800 cm, from about 1 cm to about 1700 cm, from about 1 cm to about 1600 cm, from about 1 cm to about 1500 cm, from about 1 cm to about 1400 cm, from about 1 cm to about 1300 cm, from about 1 cm to about 1200 cm, from about 1 cm to about 1100 cm, from about 1 cm to about 1000 cm, from about 1 cm to about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, or from about 1 cm to about 10 cm. In some embodiments, a shelf can have a height between shelves of from about 0 cm to about 2000 cm, from about 0 cm to about 1900 cm, from about 0 cm to about 1800 cm, from about 0 cm to about 1700 cm, from about 0 cm to about 1600 cm, from about 0 cm to about 1500 cm, from about 0 cm to about 1400 cm, from about 0 cm to about 1300 cm, from about 0 cm to about 1200 cm, from about 0 cm to about 1100 cm, from about 0 cm to about 1000 cm, from about 0 cm to about 900 cm, from about 0 cm to about 800 cm, from about 0 cm to about 700 cm, from about 0 cm to about 600 cm, from about 0 cm to about 500 cm, from about 0 cm to about 400 cm, from about 0 cm to about 300 cm, from about 0 cm to about 200 cm, from about 0 cm to about 100 cm, from about 0 cm to about 90 cm, from about 0 cm to about 80 cm, from about 0 cm to about 70 cm, from about 0 cm to about 60 cm, from about 0 cm to about 50 cm, from about 0 cm to about 40 cm, from about 0 cm to about 30 cm, from about 0 cm to about 20 cm, from about 0 cm to about 10 cm, from about 0 cm to about 9 cm, from about 0 cm to about 8 cm, from about 0 cm to about 7 cm, from about 0 cm to about 6 cm, from about 0 cm to about 5 cm, from about 0 cm to about 4 cm, from about 0 cm to about 3 cm, from about 0 cm to about 2 cm, from about 0 cm to about 1 cm, from about 0 cm to about 0.9 cm, from about 0 cm to about 0.8 cm, from about 0 cm to about 0.7 cm, from about 0 cm to about 0.6 cm, from about 0 cm to about 0.5 cm, from about 0 cm to about 0.4 cm, from about 0 cm to about 0.3 cm, from about 0 cm to about 0.2 cm, or from about 0 cm to about 0.1 cm.

In some embodiments, a rack, a container, or a shelf can be constructed of any material. For example, a rack, a shelf, or a container may be constructed from in whole or in part from polypropylene, polypropylene co-polymers, polyethylene, polyester, polystyrene, polycarbonate, polysulfone, polyolefin, polyetherimide, fluorinated ethylene propylene, polyphenylsulfone, polyetheretherketone, perfluoroalkoxy, ethylene tetrafluoroethylene, ethylene chlorotrifluoroethylene, polychlorotrifluoroethylene, polytetrafluoroethylene, polyphenyl sulfide, silicone, silicone co-polymers, stainless steel, steel alloys, aluminum, aluminum alloys, titanium, titanium alloys, glass, ceramic, or any combination thereof. In some cases, a container can be at least partially recyclable. In some cases, a container can be at fully recyclable. In some cases, a container can be at least partially biodegradable. In some cases, a container can be fully biodegradable. In some cases, a container can be reusable. In some cases, a container can be a single-use bioprocessing container. In some cases, a container can be a single-use, gas permeable bag (e.g., for the growth of cells).

In some cases, a component of a system (e.g. a rack, a container, a shelf, a tube, a pipe, etc) can be sterilizable. In some cases, a component can be sterilized using heat. For example, a component can be flame sterilized, autoclaved, or otherwise contacted with heat. In some cases, a component can be sterilized by steam. For example, a component can be autoclaved, steamed-in-place (SIP), or otherwise contacted with steam. In some cases, a component can be sterilized by irradiation. Such an irradiation can include, for example, irradiation with an ultraviolet, microwave, or gamma source. In some cases, a component can be sterilized using a chemical sterilization. For example, a component can be sterilized by ethylene oxide, vaporized hydrogen peroxide, or otherwise contacted with chemicals. A chemical sterilization can include contacting a component with a chemical entity capable of at least partially reducing a microorganism population on the component. Examples of such chemical entities can include an antibiotic such as Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or a salt of any of these; an antiviral compound such as Acyclovir, Brivudine, Docosanol, Famciclovir, Idoxuridine, Penciclovir, Trifluridine, Valacyclovir, Amantadine, Rimantadine, a neuraminidase inhibitor, Oseltamivir, Zanamivir, or a salt of any of these; an antifungal agent such as antifungal agents such as ciclopirox olamine, haloprogin, tolnaftate, undecylenate, topical nysatin, amorolfine, butenafine, naftifine, terbinafine; a surfactant such as polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, sugar esters of fatty acids, or glycerides of fatty acids; a quaternary ammonium compound such as benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride or domiphen bromide; small molecules such as imidazole, indoles, nitric oxide, triazoles, phenols, sulfides, polysaccharides, furanones, and bromopyrroles; amino acids and their derivatives such as L-leucine, or cysteamine.

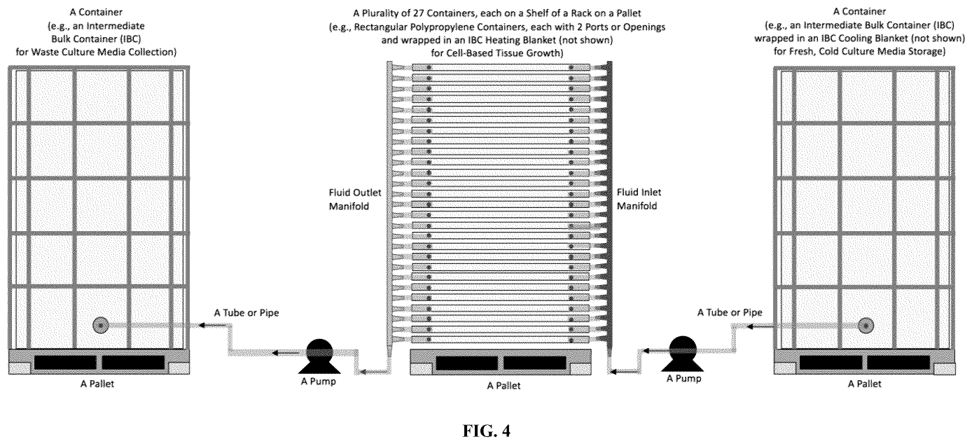

Referring to FIG. 4, in some exemplary embodiments at least one tube, pipe or opening can connect a first container to one or more other containers. A tube, opening or pipe can have a length of from about 1 cm to about 10000 cm, from about 1 cm to about 9000 cm, from about 1 cm to about 8000 cm, from about 1 cm to about 7000 cm, from about 1 cm to about 6000 cm, from about 1 cm to about 5000 cm, from about 1 cm to about 4000 cm, from about 1 cm to about 3000 cm, from about 1 cm to about 2000 cm, from about 1 cm to about 1000 cm, from about 1 cm to about 900 cm, from about 1 cm to about 800 cm, from about 1 cm to about 700 cm, from about 1 cm to about 600 cm, from about 1 cm to about 500 cm, from about 1 cm to about 400 cm, from about 1 cm to about 300 cm, from about 1 cm to about 200 cm, from about 1 cm to about 100 cm, from about 1 cm to about 90 cm, from about 1 cm to about 80 cm, from about 1 cm to about 70 cm, from about 1 cm to about 60 cm, from about 1 cm to about 50 cm, from about 1 cm to about 40 cm, from about 1 cm to about 30 cm, from about 1 cm to about 20 cm, from about 1 cm to about 10 cm, from about 1 cm to about 9 cm, from about 1 cm to about 8 cm, from about 1 cm to about 7 cm, from about 1 cm to about 6 cm, from about 1 cm to about 5 cm, from about 1 cm to about 4 cm, from about 1 cm to about 3 cm, or from about 1 cm to about 2 cm, A tube or pipe can have an inside diameter of from about 0.1 cm to about 1000 cm, from about 0.1 cm to about 900 cm, from about 0.1 cm to about 800 cm, from about 0.1 cm to about 700 cm, from about 0.1 cm to about 600 cm, from about 0.1 cm to about 500 cm, from about 0.1 cm to about 400 cm, from about 0.1 cm to about 300 cm, from about 0.1 cm to about 200 cm, from about 0.1 cm to about 100 cm, from about 0.1 cm to about 90 cm, from about 0.1 cm to about 80 cm, from about 0.1 cm to about 70 cm, from about 0.1 cm to about 60 cm, from about 0.1 cm to about 50 cm, from about 0.1 cm to about 40 cm, from about 0.1 cm to about 30 cm, from about 0.1 cm to about 20 cm, from about 0.1 cm to about 10 cm, from about 0.1 cm to about 9 cm, from about 0.1 cm to about 8 cm, from about 0.1 cm to about 7 cm, from about 0.1 cm to about 6 cm, from about 0.1 cm to about 5 cm, from about 0.1 cm to about 4 cm, from about 0.1 cm to about 3 cm, from about 0.1 cm to about 2 cm, or from about 0.1 cm to about 1 cm. A tube of a pipe can have a wall thickness of from about 0.01 cm to about 10 cm, from about 0.01 cm to about 9 cm, from about 0.01 cm to about 8 cm, from about 0.01 cm to about 7 cm, from about 0.01 cm to about 6 cm, from about 0.01 cm to about 5 cm, from about 0.01 cm to about 4 cm, from about 0.01 cm to about 3 cm, from about 0.01 cm to about 2 cm, from about 0.01 cm to about 1 cm, from about 0.01 cm to about 0.9 cm, from about 0.01 cm to about 0.8 cm, from about 0.01 cm to about 0.7 cm, from about 0.01 cm to about 0.6 cm, from about 0.01 cm to about 0.5 cm, from about 0.01 cm to about 0.4 cm, from about 0.01 cm to about 0.3 cm, from about 0.01 cm to about 0.2 cm, or from about 0.01 cm to about 0.1 cm. In some embodiments, one or more pumps can be utilized to transfer fluid from at least one container to at least another container or component of a system through the tubes, openings or pipes. Such a fluid can include a medium (e.g. growth medium), a pH buffer solution, water, or other aqueous solution used in cell culture. In some embodiments, the tubes, openings or pipes can carry gases (e.g., oxygen, carbon dioxide). In some embodiments, the tubes, openings or pipes can carry solids (e.g., powdered growth medium, powdered growth medium supplements, cell-culture microcarriers).

In some embodiments, a tube, opening or pipe can be used to exchange a growth medium during a cell culturing. In some cases, a culture medium can become depleted during the growth of cell culture. In some cases, spent medium can be removed along with dead cells from a container using a tube or pipe operatively connected to a pump, and fresh growth medium can be pumped into the container. In some cases, exchange of new media can occur simultaneously, or after a removal of spent media. In some cases, an exchange can occur over a period of time. In some cases, an exchange can offer at least every 4, 8, 12, 24, 36, 48, or 72 hours. In some cases, an exchange can occur on demand by a user of a system by activation of a pump. In some cases, an exchange can occur continuously. In some cases, fluid removal or addition can be mediated or assisted by gravity.

### Motion Control System

A system described herein can include a motion control system. As used herein, the term “motion control system” can refer to a component that is configured to translate a component of a system described herein (e.g. a container, a rack, a shelf, or the like) a certain distance over a certain time. By using such a system, items placed in a container may be translated across the container. In some cases, this can create a dynamic flow across a container. As described herein, such a dynamic flow can produce a more viable cell culture as compared to a system in which a dynamic flow does not occur (i.e. in a conventional static cell culture system).

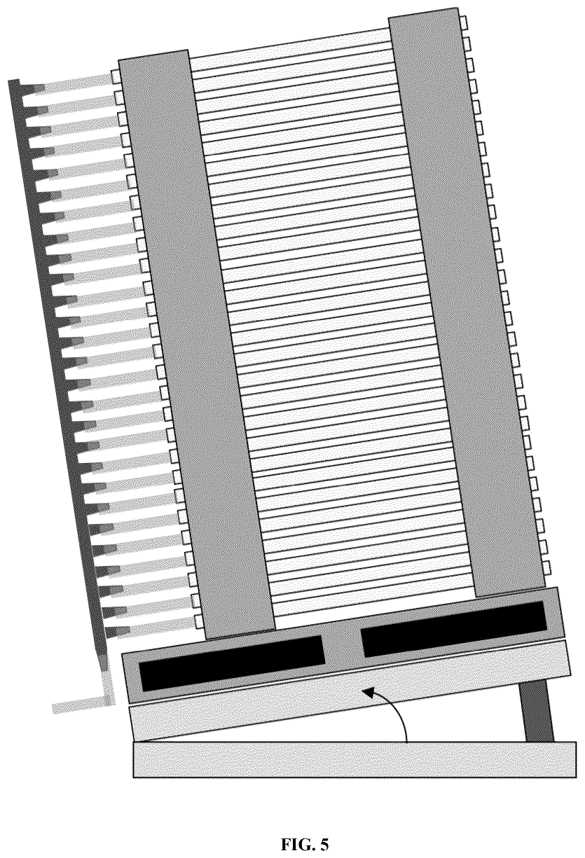

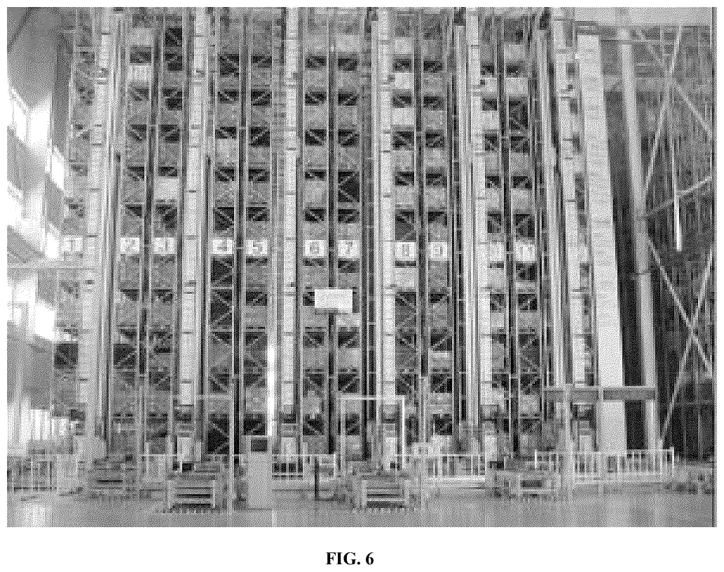

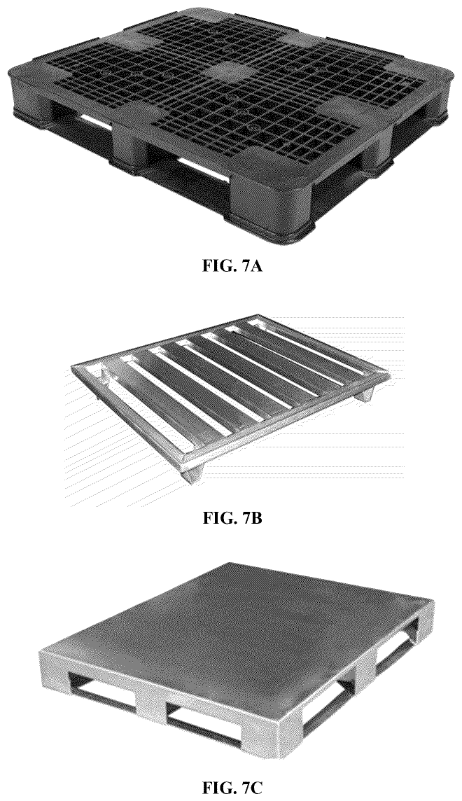

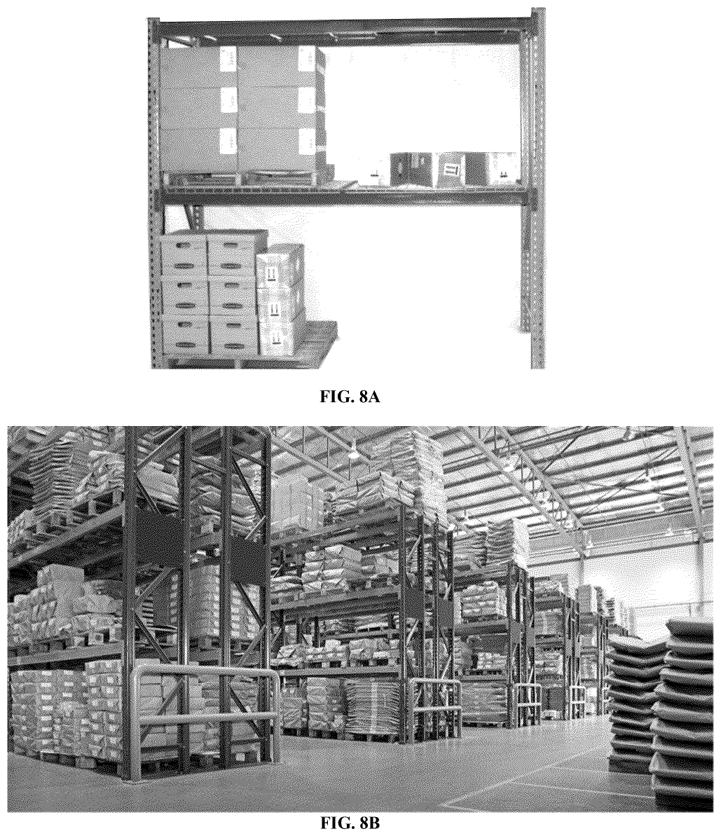

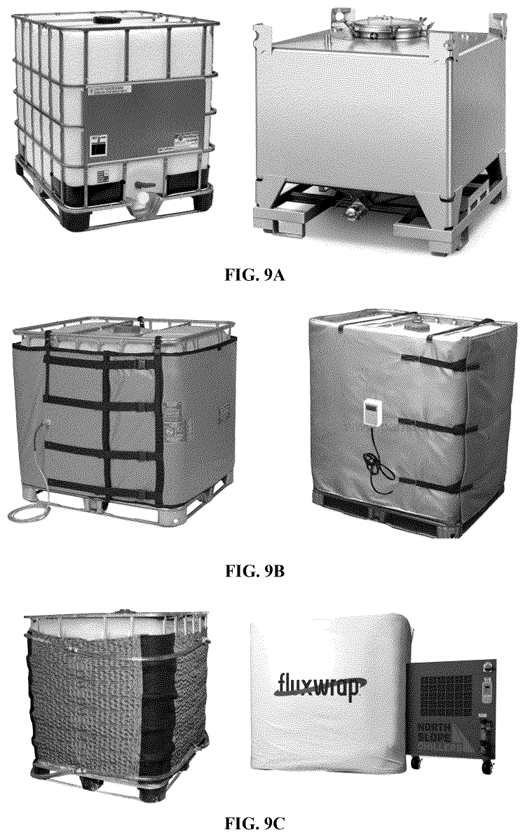

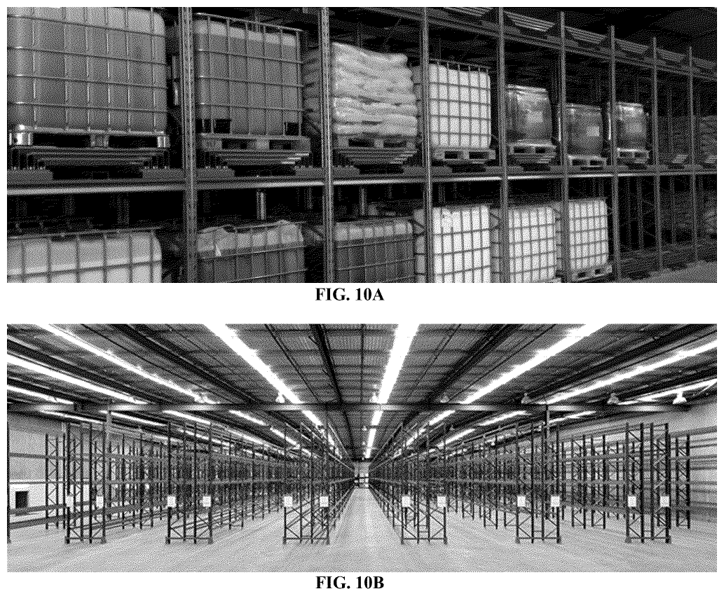

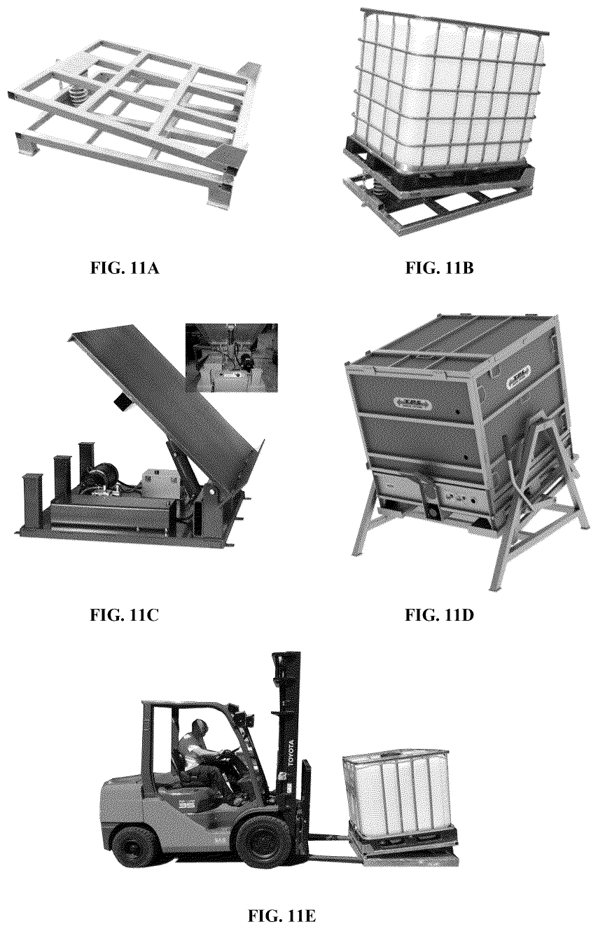

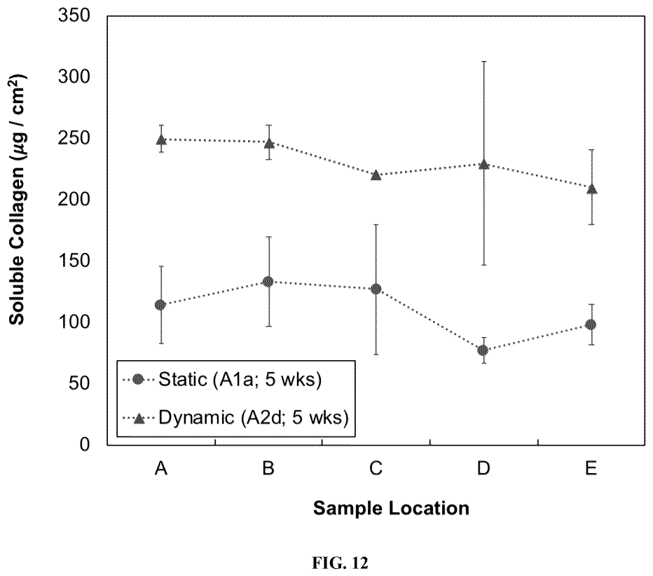

Referring to FIG. 5 and FIG. 12, in some exemplary embodiments a system can comprise a motion control system which can, due to container tilting, rotating or rocking, elicit an increase in extracellular matrix synthesis or accretion by a cell culture (e.g., increased collagen content). Referring to FIG. 4, in an exemplary embodiment, at least one rack, shelf, or container can be configured on a pallet and a pallet tilter or pallet tilter-like motion control system. Such tilting and rocking can improve cell viability of a cell culture in a system described herein, relative to a static system that does not utilize a motion control system. Furthermore, a system as described herein employing a motion control system can improve expression of a product (e.g. a marker) within a cell culture, as compared to a comparable static cell culture bioreactor. For example, FIG. 12 illustrates an improvement in production of collagen in a mammalian cell culture grown on a system as described herein comprising a motion control system, as compared to a comparable static bioreactor. As shown in FIG. 12, a cell culture grown in a system as described herein employing shacking or rocking produced approximately 250 μg/cm² of collagen at 5 weeks, compared to approximately 100 μg/cm² of collagen produced in a static bioreactor. This increased production when cultured in a dynamic system as described herein represents a surprising and unexpected improvement relative to standard static bioreactors. Furthermore, this approach is unconventional in that mammalian cell cultures are typically grown in static incubators, where the conventional wisdom would be to avoid turbulence during cell growth. Accordingly, a skilled artisan would not have expected a system as described herein, which employs tilting or rocking via a motion control system, to improve production of an accretion such as collagen.

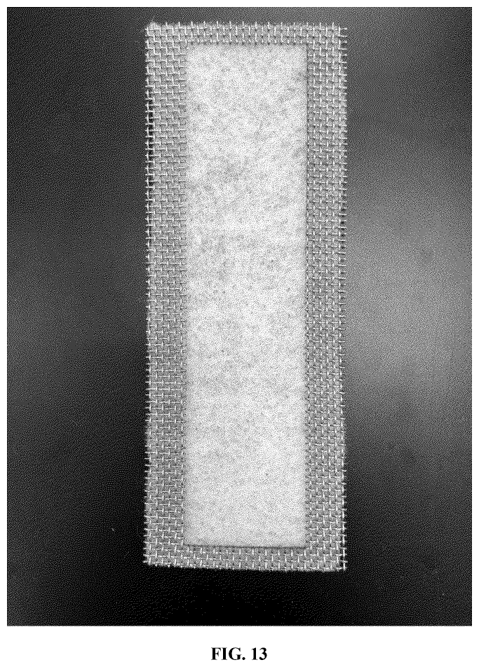

A system may further comprise a cell growth substrate. Such a cell growth substrate can be placed, attached, etched, etc, into a container described herein. FIG. 13 depicts an exemplary cell growth substrate. A cell growth substrate can have a substantially two dimensional or a substantially three-dimensional shape. In some embodiments, a cell growth substrate can be constructed from a material selected from the group consisting of: polystyrene, polyester, polyethylene terephlatate, poly(glycolic acid), poly(lactic acid), poly(lactic-co-glycolic acid), poly(ethylene glycol), polyurethane, poly(glycerol sebacate), polycarbonate, polyetherimide, stainless steel, silver, gold, platinum, palladium, iridium, titanium, tantalum, noble metal, collagen, fibrin, silk, wool, wood, hemp, linin, cotton, cellulose, grass, leaves, straw, lignin, chitosan, chitosan derivatives, diamond, sapphire, amethyst, ruby, emerald, opal, topaz, quartz, marble, slate, lava rock, coral, sponge, diatom, sand dollar, sea shell, starfish, seaweed, kelp, algae, a scaffold disclosed herein or any combination thereof. In some cases, a cell growth substrate can be constructed of a material such as lunar dust, Martian dust, or other components derived from Earth or other planetary bodies or their satellites, meteors, meteorites, asteroids, comets, or other components derived from space.

Any system capable of tilting or rocking a container can be utilized. For example, an intermediate bulk container tilter or similar motion control device can be used. Such a motion control system can be configured to, when actuated or activated, rock or tilt a container.

A motion control system can rock or tilt a component of a system, such that the component is translated over a certain distance. For example, a motion control system can translate a component at least 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, or 1000 cm. In some embodiments, a motion control device can be configured to rock or tilt a container at an angle of greater than about 0 degrees to about 360 degrees. For example, a motion control device can rock or tilt a container about 1 degree, about 2 degrees, about 3 degrees, about 4 degrees, about 5 degrees, about 6 degrees, about 7 degrees, about 8 degrees, about 9 degrees, about 10 degrees, about 11 degrees, about 12 degrees, about 13 degrees, about 14 degrees, about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, about 45 degrees, about 46 degrees, about 47 degrees, about 48 degrees, about 49 degrees, about 50 degrees, about 51 degrees, about 52 degrees, about 53 degrees, about 54 degrees, about 55 degrees, about 56 degrees, about 57 degrees, about 58 degrees, about 59 degrees, about 60 degrees, about 61 degrees, about 62 degrees, about 63 degrees, about 64 degrees, about 65 degrees, about 66 degrees, about 67 degrees, about 68 degrees, about 69 degrees, about 70 degrees, about 71 degrees, about 72 degrees, about 73 degrees, about 74 degrees, about 75 degrees, about 76 degrees, about 77 degrees, about 78 degrees, about 79 degrees, about 80 degrees, about 81 degrees, about 82 degrees, about 83 degrees, about 84 degrees, about 85 degrees, about 86 degrees, about 87 degrees, about 88 degrees, about 89 degrees, about 90 degrees, about 91 degrees, about 92 degrees, about 93 degrees, about 94 degrees, about 95 degrees, about 96 degrees, about 97 degrees, about 98 degrees, about 99 degrees, about 100 degrees, about 101 degrees, about 102 degrees, about 103 degrees, about 104 degrees, about 105 degrees, about 106 degrees, about 107 degrees, about 108 degrees, about 109 degrees, about 110 degrees, about 111 degrees, about 112 degrees, about 113 degrees, about 114 degrees, about 115 degrees, about 116 degrees, about 117 degrees, about 118 degrees, about 119 degrees, about 120 degrees, about 121 degrees, about 122 degrees, about 123 degrees, about 124 degrees, about 125 degrees, about 126 degrees, about 127 degrees, about 128 degrees, about 129 degrees, about 130 degrees, about 131 degrees, about 132 degrees, about 133 degrees, about 134 degrees, about 135 degrees, about 136 degrees, about 137 degrees, about 138 degrees, about 139 degrees, about 140 degrees, about 141 degrees, about 142 degrees, about 143 degrees, about 144 degrees, about 145 degrees, about 146 degrees, about 147 degrees, about 148 degrees, about 149 degrees, about 150 degrees, about 151 degrees, about 152 degrees, about 153 degrees, about 154 degrees, about 155 degrees, about 156 degrees, about 157 degrees, about 158 degrees, about 159 degrees, about 160 degrees, about 161 degrees, about 162 degrees, about 163 degrees, about 164 degrees, about 165 degrees, about 166 degrees, about 167 degrees, about 168 degrees, about 169 degrees, about 170 degrees, about 171 degrees, about 172 degrees, about 173 degrees, about 174 degrees, about 175 degrees, about 176 degrees, about 177 degrees, about 178 degrees, about 179 degrees, about 180 degrees, about 181 degrees, about 182 degrees, about 183 degrees, about 184 degrees, about 185 degrees, about 186 degrees, about 187 degrees, about 188 degrees, about 189 degrees, about 190 degrees, about 191 degrees, about 192 degrees, about 193 degrees, about 194 degrees, about 195 degrees, about 196 degrees, about 197 degrees, about 198 degrees, about 199 degrees, about 200 degrees, about 201 degrees, about 202 degrees, about 203 degrees, about 204 degrees, about 205 degrees, about 206 degrees, about 207 degrees, about 208 degrees, about 209 degrees, about 210 degrees, about 211 degrees, about 212 degrees, about 213 degrees, about 214 degrees, about 215 degrees, about 216 degrees, about 217 degrees, about 218 degrees, about 219 degrees, about 220 degrees, about 221 degrees, about 222 degrees, about 223 degrees, about 224 degrees, about 225 degrees, about 226 degrees, about 227 degrees, about 228 degrees, about 229 degrees, about 230 degrees, about 231 degrees, about 232 degrees, about 233 degrees, about 234 degrees, about 235 degrees, about 236 degrees, about 237 degrees, about 238 degrees, about 239 degrees, about 240 degrees, about 241 degrees, about 242 degrees, about 243 degrees, about 244 degrees, about 245 degrees, about 246 degrees, about 247 degrees, about 248 degrees, about 249 degrees, about 250 degrees, about 251 degrees, about 252 degrees, about 253 degrees, about 254 degrees, about 255 degrees, about 256 degrees, about 257 degrees, about 258 degrees, about 259 degrees, about 260 degrees, about 261 degrees, about 262 degrees, about 263 degrees, about 264 degrees, about 265 degrees, about 266 degrees, about 267 degrees, about 268 degrees, about 269 degrees, about 270 degrees, about 271 degrees, about 272 degrees, about 273 degrees, about 274 degrees, about 275 degrees, about 276 degrees, about 277 degrees, about 278 degrees, about 279 degrees, about 280 degrees, about 281 degrees, about 282 degrees, about 283 degrees, about 284 degrees, about 285 degrees, about 286 degrees, about 287 degrees, about 288 degrees, about 289 degrees, about 290 degrees, about 291 degrees, about 292 degrees, about 293 degrees, about 294 degrees, about 295 degrees, about 296 degrees, about 297 degrees, about 298 degrees, about 299 degrees, about 300 degrees, about 301 degrees, about 302 degrees, about 303 degrees, about 304 degrees, about 305 degrees, about 306 degrees, about 307 degrees, about 308 degrees, about 309 degrees, about 310 degrees, about 311 degrees, about 312 degrees, about 313 degrees, about 314 degrees, about 315 degrees, about 316 degrees, about 317 degrees, about 318 degrees, about 319 degrees, about 320 degrees, about 321 degrees, about 322 degrees, about 323 degrees, about 324 degrees, about 325 degrees, about 326 degrees, about 327 degrees, about 328 degrees, about 329 degrees, about 330 degrees, about 331 degrees, about 332 degrees, about 333 degrees, about 334 degrees, about 335 degrees, about 336 degrees, about 337 degrees, about 338 degrees, about 339 degrees, about 340 degrees, about 341 degrees, about 342 degrees, about 343 degrees, about 344 degrees, about 345 degrees, about 346 degrees, about 347 degrees, about 348 degrees, about 349 degrees, about 350 degrees, about 351 degrees, about 352 degrees, about 353 degrees, about 354 degrees, about 355 degrees, about 356 degrees, about 357 degrees, about 358 degrees, about 359 degrees, or about 360 degrees.

A motion control system can be configured to rock, rotate or tilt at a certain frequency. For example, a motion control system can be configured to rock, rotate or tilt at a frequency of from about 0.1 to about 2 Hz, from about 0.2 to about 2 Hz, from about 0.3 to about 2 Hz, from about 0.4 to about 2 Hz, from about 0.5 to about 2 Hz, from about 0.6 to about 2 Hz, from about 0.7 to about 2 Hz, from about 0.8 to about 2 Hz, from about 0.9 to about 2 Hz, from about 1 to about 2 Hz, from about 0.1 Hz to about 10 Hz, from about 0.2 to about 10 Hz, from about 0.3 to about 10 Hz, from about 0.4 to about 10 Hz, from about 0.5 to about 10 Hz, from about 0.6 to about 10 Hz, from about 0.7 to about 10 Hz, from about 0.8 to about 10 Hz, from about 0.9 to about 10 Hz, from about 1 to about 10 Hz, from about 2 to about 10 Hz, from about 3 to about 10 Hz, from about 4 to about 10 Hz, from about 5 to about 10 Hz, from about 10 to about 100 Hz, from about 20 Hz to about 2000 Hz, from about 50 Hz to about 1000 Hz, or from about 100 Hz to about 500 Hz. In some embodiments, a user can program a motion control system to rock, rotate, or tilt at a particular distance, at a particular tilt angle, at a particular frequency, or any combination thereof.

### Monitoring system

In some embodiments, a monitoring system can be used in a container. In some embodiments, a monitoring system can monitor a temperature, a humidity, a medium level, a concentration of a component of a media, a time, a gaseous concentration, or any combination thereof. In some embodiments, a monitoring system can transmit instructions to another component of a system. In some embodiments, a monitoring system can detect damage to a component of a system. In some embodiments, a monitoring system can detect a leak. In some embodiments, a monitoring system can alert a user that an action must be taken. In some embodiments, a monitoring system can comprise, a sensor, a camera, or a combination thereof. In some embodiments, a sensor can comprise a thermistor, a thermometer, a pH sensor, a humidity sensor, a pressure sensor, a smoke detector, or any combination thereof.

In some embodiments, a monitoring system can interact with a control system. In some embodiments, a monitoring system can trigger a control system. In some embodiments, a control system can control a temperature, a humidity, a medium level, a time, a gaseous concentration, or any combination thereof. In some embodiments, a low level of medium can trigger a release of more medium from a reservoir to a medium.

### Temperature Control

In some embodiments, a temperature control system can be utilized to maintain a container at a specified temperature. For example, a temperature may represent: an optimal, species-specific temperature for cell growth (e.g., 37 degrees Celsius); 2-8 degrees Celsius for storage of fluid media; −20 degrees Celsius for storage of frozen fluid media; 200 degrees Celsius for dry heat sterilization. As disclosed herein, any temperature control system or combination of temperature control systems may be utilized as a component of the scalable bioreactor system and methods for tissue engineering disclosed herein, including any conventional refrigeration or freezer technology, any Peltier-based cooling technologies, any incubator technologies, any HVAC technologies, any heating or cooling jacket technologies, any geothermal technologies, any natural heating or cooling technologies (e.g., direct sunlight, a hot house, a cold cellar). In an exemplary embodiment, one or more containers are heating using an intermediate bulk container (IBC) heating blanket or jacket. In some embodiments, a temperature can be automatically adjusted based on a sensor and a programmed temperature. In some embodiments, a temperature can be adjusted based a growth of a cell culture.

In some embodiments, a humidity control system can be utilized to maintain a specified humidity level. Such a system can utilize input of water vapor to maintain a set humidity. Alternatively, a water pan can be placed in the system to generate humidity. In some cases, a cell culturing in a system described herein can be conducted with a relative humidity of 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative humidity. In some embodiments, a humidity can be automatically adjusted based on a sensor and a programmed humidity. In some embodiments, humidity can be adjusted based a growth of a cell culture.

A system as described herein can be operatively coupled to at least one power source. For example, any component of a system described herein (i.e. a motion control system, a temperature control system, etc) be collectively or separately connected to a power source to power each component.

### Cell Culture and Synthetic Leather

Any cell, including animal cells can be grown in a system described herein. For example, an animal cell can be derived from human and non-human mammals, such as non-human primates and members of the bovine, ovine, porcine, equinine, canine and feline species as well as rodents such as mice, rats and guinea pigs, members of the lagomorph family including rabbit, fish including shark and stingray, birds including ostrich and reptiles including lizards, snakes and crocodiles.

In some cases, a cell-based tissue can be produced in a system as described herein by culturing a cell culture in the system. Such a tissue can be used to generate, for example, a synthetic leather. In some embodiments, cells and/or tissues cultured in a system descried herein can be comprised in synthetic leather. In some embodiments, synthetic leather, or a portion thereof can be cultured in a system described herein. In some embodiments, a cell culture or a cultured tissue can comprise a transgene, a heterologous RNA, or an epigenetically modified base. In some embodiments, an epigenetically modified base can be methylated, hydroxymethylated, carboxylated, or formylated.

A synthetic leather can comprise one or more cell layers. A synthetic leather can comprise one or more cell layers and the cell layers or part thereof can be cultured or grown in a system descried herein. For example, a synthetic leather can comprise one or more of: a dermal layer, an epidermal layer, a basement membrane or a basement membrane substitute. In some embodiments, a dermal layer, an epidermal layer, a basement membrane or a basement membrane substitute or a portion thereof can be cultured or grown in a system described herein. In some embodiments, a synthetic leather may not comprise one or more of: a dermal layer, an epidermal layer, a basement membrane or a basement membrane substitute. A synthetic leather can further comprise hypodermis, scale, scute, osteoderm, or a combination thereof. In some cases, a synthetic layer can can comprise a full thickness skin equivalent. Such full thickness skin equivalent can comprise any one or combination of the layers disclosed herein. In some embodiments, a full thickness equivalent may not comprise one or more of: a dermal layer, an epidermal layer, a basement membrane or a basement membrane substitute. In some embodiments, a portion of one or more cell layers in a synthetic leather can be removed, e.g., by shaving. In some cases, a synthetic leather can be tanned. The tanning can be performed after formation of one or more of the cell layers or layered structures. The tanning can be performed after at least a portion of a cell layer can be removed from a synthetic leather. In some cases, a synthetic leather can be further processed. In some cases, a synthetic leather can comprise a hair follicle cell and/or a melanocyte. The hair follicle cell and/or the melanocyte can be differentiated from a stem cell (e.g., an iPSC).

In some embodiments, a tanned synthetic leather can comprise a layered structure. In some embodiments, a layered structure or portions thereof can be cultured or grown in a system described herein. A layered structure can comprise an artificial dermal layer comprising a fibroblast. A layered structure can also comprise an artificial epidermal layer comprising a keratinocyte. In some cases, a layered structure can comprise an artificial dermal layer comprising a fibroblast and an artificial epidermal layer comprising a keratinocyte. In some cases, a fibroblast or a keratinocyte can be differentiated from an induced pluripotent stem cell.

In some cases, a tanned synthetic leather can comprise at least part of a dermal layer. In some cases, a tanned synthetic leather does not comprise a dermal layer. In some cases, a dermal layer can be removed.

### Dermal Layer

A synthetic leather can comprise a dermal layer (e.g., an artificial dermal layer). A dermal layer can be an engineered dermis equivalent, e.g., an artificial dermal layer formed in vitro. In some embodiments, a dermal layer or parts thereof can be cultured or grown in a system described herein.

A dermal layer can comprise cells of connective tissue. For example, a dermal layer can comprise fibroblasts. Fibroblasts in the dermal layer can express one or more markers including, but not limited to, cluster of differentiation 10 (CD10), cluster of differentiation 73 (CD73), cluster of differentiation 44 (CD44), cluster of differentiation 90 (CD90), type I collagen, type III collagen, and prolyl-4-hydroxylase beta fibroblasts. In some cases, a dermal layer also can comprise other types of cells, such as immune cells, macrophages, adipocytes, or a combination thereof.

A dermal layer can further comprise matrix components in addition to cells. Examples of matrix components include but are not limited to any one or more of collagen, elastin, and extrafibrillar matrix, an extracellular gel-like substance primarily composed of glycosaminoglycans (e.g., hyaluronan), proteoglycans, and glycoproteins.

A dermal layer can comprise a matrix support. A matrix support can be a scaffold. The matrix support can comprise contracted collagen gels. Alternatives to a pure collagen matrix can be a polyglygolic acid mesh, or collagen and glycosaminoglycan matrix covered with a silastic membrane (C-GAG) or various biopolymers, e.g. chitosan. In some cases, the matrix can be seeded with fibroblasts, e.g., to give rise to organotypic models. Naturally derived dermis, from allogenic cadaver skin can also be used with keratinocyte sheets. A variation of this technique can use lyophilized devitalized dermis from cadaver skin to support the keratinocyte sheets.

The thickness of leather units may be reported in millimeters, ounces, or irons. (One-ounce equals 1/64 in. or 0.0156 in. or 0.396 mm. One iron equals 1/48 in. or 0.0208 in. or 0.53 mm.)

The thickness of a dermal layer can be engineered to fit the function or use of a synthetic leather. A dermal layer can have a thickness from about 0.01 mm to about 50 mm. For example, a dermal layer can have a thickness from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a dermal layer can have a thickness from about 0.02 mm to 5 mm. For example, a dermal layer can have a thickness from about 0.1 mm to 0.5 mm. For example, a dermal layer can have a thickness from about 0.2 mm to 0.5 mm. In some cases, the thickness of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the thickness of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a thickness of at least about 50 mm.

The length of a dermal layer can be engineered to fit the function or use of a synthetic leather. A dermal layer can have a length from about 0.01 mm to about 50 m. For example, a dermal layer can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a dermal layer can have a length from about 0.02 mm to 5 mm. For example, a dermal layer can have a length from about 0.1 mm to 0.5 mm. For example, a dermal layer can have a length from about 0.2 mm to 0.5 mm. In some cases, the length of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the length of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a length of at least about 50, 60, 70, 80, 90, 100, 200, 300, 400, 500, 700, 1000 mm. In some embodiments, a dermal layer can have a length of at least about 50, 60, 70, 80, 90, 100, 200, 300, 400, 500, 600, 700 cm. In some embodiments, a dermal layer can have a length of at least about 50, 60, 70, 80, 90, 100, 200, 300, 400 m.

The width of a dermal layer can be engineered to fit the function or use of a synthetic leather. A dermal layer can have a width from about 0.01 mm to about 50 m. For example, a dermal layer can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a dermal layer can have a width from about 0.02 mm to 5 mm. For example, a dermal layer can have a width from about 0.1 mm to 0.5 mm. For example, a dermal layer can have a width from about 0.2 mm to 0.5 mm. In some cases, the width of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the width of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a width of at least about 50, 60, 70, 80, 90, 100, 200, 300, 400, 500, 700, 1000 mm. In some embodiments, a dermal layer can have a width of at least about 50, 60, 70, 80, 90, 100, 200, 300, 400, 500, 600, 700 cm. In some embodiments, a dermal layer can have a width of at least about 50, 60, 70, 80, 90, 100, 200, 300, 400 m.

A synthetic leather can comprise one or more dermal layers. For example, a synthetic leather can have at least 1, 2, 3, 4, 5, 6, 7, 8, 9, 10, 12, 14, 16, 18, 20, 40, 60, 80, or 100 dermal layers. When a synthetic leather can comprise more than one dermal layer, a dermal layer can be placed upon another dermal layer. For example, a synthetic leather can comprise two dermal layers, e.g., a first dermal layer and a second dermal layer. The first dermal layer can be placed upon the second dermal layer.

A dermal layer can be stratified, e.g., having a plurality of sublayers. The sublayers can have different compositions, e.g., different concentrations of the fibers. The sublayers of a dermal layer can have different thicknesses and densities. For example, a dermal layer can have a papillary dermal layer, a reticular dermal layer, or any combination thereof. A papillary dermal layer can comprise loose areolar connective tissue and/or loosely arranged fibers, e.g., collagen fibers. A reticular dermal layer can comprise dense irregular connective tissue, including collagen fibers and dermal elastic fibers.

A dermal layer can comprise a free collagen matrix or lattice, which can be contractile in all directions, and homogeneous. Fibroblasts, and where appropriate other types of cells of the dermis, can be distributed in a continuous collagen gel. The dermis equivalent can comprise at least one matrix of collagen type I in which the fibroblasts are distributed. It can also contain other extracellular matrix constituents. Extracellular matrix constituent can include collagens, e.g., collagen IV, laminins, entactin, fibronectin, proteoglycans, glycosaminoglycans or hyaluronic acid. A dermal layer can contain collagen type IV and laminin, entactin, or a combination thereof. The concentrations of these various constituents can be adjusted. For example, the concentration of laminin can be from about 1% to about 15% of the final volume. For example, the concentration of collagen IV can be from about 0.3% to about 4.5% of the final volume. For example, the concentration of entactin can be from about 0.05% to about 1% of the final volume. The collagen used can be collagen of bovine origin, from rat tail or from fish, or any other source of natural collagen or collagen produced by genetic engineering which allows contraction in the presence of fibroblasts. In some embodiments, collagen can be from an unnatural source. The matrix can be a gel of collagen which may not taut, obtained by contraction both horizontally and vertically, which does not impose a preferential organization of the fibroblasts. Such a matrix, also termed “free”, may not adhere to the support and the volumes thereof can be modified without limit, conferring on it a varying thickness and diameter. The thickness of the dermis equivalent can be at least 0.05 cm and, in some cases, approximately from 0.05 to 2 cm. The thickness can also be increased without harming the advantageous properties of the skin equivalent or synthetic leather. In some cases, the thickness can be from about 3 mm to about 20 cm or more.

### Epidermal Layer

A synthetic leather can comprise an epidermal layer (e.g., an artificial epidermal layer). An epidermal layer can be an engineered epidermis equivalent, e.g., an artificial epidermal layer formed in vitro. In some embodiments, an epidermal layer or parts thereof can be cultured or grown in a system described herein.

An epidermal layer can comprise one or more types of cells, including keratinocytes, melanocytes, Langerhans cells, Merkel cells, and inflammatory cells. For example, an epidermal layer can comprise keratinocytes. Keratinocytes in an epidermal layer can include epithelial keratinocytes, basal keratinocytes, proliferating basal keratinocytes, differentiated suprabasal keratinocytes, or any combination thereof.

In some cases, an epidermal layer can comprise at least basal keratinocytes, e.g., keratinocytes which are not differentiated. An epidermal layer can further comprise partially differentiated keratinocytes as well as fully differentiated keratinocytes. In one or more epidermal layers in a synthetic leather there can be a transition from undifferentiated basal keratinocytes to fully differentiated keratinocytes as one progresses from the dermal-epidermal junction where the basal keratinocytes are localized.

Basal keratinocytes can express hemidesmosomes, which serve to help secure the epidermal and dermal layers together. Basal keratinocytes can also serve to regenerate skin. An epidermal layer in a synthetic leather herein can have basal keratinocytes that serve these functions. Thus, a synthetic leather comprising such basal keratinocytes can be capable of regeneration. In some embodiments, both E- and P-cadherin's are present in epidermal keratinocytes along the basal membrane zone (BMZ). In some embodiments, keratinocytes which are differentiated and located away from the BMZ only express E-cadherin.

The basal keratinocytes of an epidermal layer can be aligned in a layer in direct contact with the dermal layer, serving as the boundary between the differentiated keratinocytes and the fibroblasts. In alternative cases, there are gaps between the basal keratinocytes and the dermal layer. Still further, there can be gaps between the basal keratinocytes and other basal keratinocytes, leaving gaps between the differentiated keratinocytes and the dermal layer. In these latter cases where there are gaps between the basal or differentiated keratinocytes and the dermal layer, the dermal and epidermal layers are not uniformly in contact with one another but are adjacent to each other. They are adjacent in that there can be generally fluid, but substantially no other intervening materials such as layers of cells, collagen, matrices or other supports between the dermal and epidermal layers.

Keratinocytes in an epidermal layer can express one or more markers. Such markers include, but are not limited to, Keratin 14 (KRT14), tumor protein p63 (p63), Desmoglein 3 (DSG3), Integrin, beta 4 (ITGB4), Laminin, alpha 5 (LAMA5), Keratin 5 (KRT5), an isoform of tumor protein p63 (e.g., TAp63), Laminin, beta 3 (LAMB3), and Keratin 18 (KRT18).

The thickness of an epidermal layer can be engineered to fit the function or use of the synthetic leather. An epidermal layer can have a thickness from about 0.001 mm to about 10 mm. For example, an epidermal layer can have a thickness from about 0.005 mm to about 10 mm, from about 0.005 mm to about 5 mm, from about 0.005 mm to about 2 mm, from about 0.01 mm to about 10 mm, from about 0.01 mm to about 5 mm, from about 0.01 mm to about 2 mm, from about 0.01 mm to about 1, from about 0.01 mm to about 0.8 mm, from about 0.01 mm to about 0.4 mm, from about 0.01 mm to about 0.2 mm, from about 0.01 mm to about 0.1 mm, from about 0.05 mm to about 0.4 mm, from about 0.05 mm to about 0.2 mm, from about 0.05 mm to about 0.1 mm, from about 0.1 mm to about 0.4 mm, from about 0.1 mm to about 0.2 mm, from about 0.08 mm to about 1 mm, or from about 0.05 mm to about 1.5 mm. For example, an epidermal layer can have a thickness from about 0.01 mm to about 2 mm. For example, an epidermal layer can have a thickness from about 0.1 mm to about 0.22 mm. In some cases, the thickness of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the thickness of the dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some cases, thickness values described herein can be the thickness of an epidermal layer and a basement membrane substitute.

The length of an epidermal layer can be engineered to fit the function or use of a synthetic leather. An epidermal layer can have a length from about 0.01 mm to about 50 m. For example, an epidermal layer can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, an epidermal layer can have a length from about 0.02 mm to 5 mm. For example, an epidermal layer can have a length from about 0.1 mm to 0.5 mm. For example, an epidermal layer can have a length from about 0.2 mm to 0.5 mm. In some cases, the length of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the length of an epidermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, an epidermal layer can have a length of at least about 50, 60, 70, 80, 90, 100, 200, 300, 400, 500, 700, 1000 mm. In some embodiments, an epidermal layer can have a length of at least about 50, 60, 70, 80, 90, 100, 200, 300, 400, 500, 600, 700 cm. In some embodiments, an epidermal layer can have a length of at least about 50, 60, 70, 80, 90, 100, 200, 300, 400 m.

The width of an epidermal layer can be engineered to fit the function or use of a synthetic leather. An epidermal layer can have a width from about 0.01 mm to about 50 m. For example, an epidermal layer can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, an epidermal layer can have a width from about 0.02 mm to 5 mm. For example, an epidermal layer can have a width from about 0.1 mm to 0.5 mm. For example, an epidermal layer can have a width from about 0.2 mm to 0.5 mm. In some cases, the width of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the width of an epidermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, an epidermal layer can have a width of at least about 50, 60, 70, 80, 90, 100, 200, 300, 400, 500, 700, 1000 mm. In some embodiments, an epidermal layer can have a width of at least about 50, 60, 70, 80, 90, 100, 200, 300, 400, 500, 600, 700 cm. In some embodiments, an epidermal layer can have a width of at least about 50, 60, 70, 80, 90, 100, 200, 300, 400 m.

A synthetic leather can comprise one or more epidermal layers. For example, a synthetic leather can have at least about 1, 2, 3, 4, 5, 6, 7, 8, 9, 10, 12, 14, 16, 18, 20, 40, 60, 80, or 100 epidermal layers. When a synthetic leather can comprise more than one epidermal layer, one epidermal layer can be placed upon another epidermal layer. For example, a synthetic leather can comprise two epidermal layers, e.g., a first epidermal layer and a second epidermal layer. The first epidermal layer can be placed upon the second epidermal layer.

An epidermal layer can be stratified, e.g., having a plurality of sublayers. The sublayers can have different cell compositions, e.g., different types of keratinocytes. The sublayers of an epidermal layer can have different thicknesses and/or densities. For example, an epidermal layer can have one or more of cornified layer (stratum corneum), clear/translucent layer (stratum lucidum), granular layer (stratum granulosum), spinous layer (stratum spinosum), basal/germinal layer (stratum basale/germinativum), or any combination thereof. In some cases, an epidermal layer can comprise functional epidermal permeability barrier (e.g., organized lipid bilayers in stratum corneum). In some cases, a stratum corneum, stratum lucidum, stratum granulosum, stratum spinosum, or stratum basale/germinativum, can have a thickness of about 0.0001 mm to about 5 mm. In some cases, a stratum corneum, stratum lucidum, stratum granulosum, stratum spinosum, or stratum basale/germinativum, can have a thickness of at least about 0.001 mm, 0.01 mm, 0.02 mm, 0.04 mm, 0.08 mm, 0.1 mm, 0.15 mm, 0.2 mm, 0.4 mm, 0.8 mm, 1 mm, 2 mm, 4 mm, 8 mm, or 10 mm. In some cases, a stratum corneum, stratum lucidum, stratum granulosum, stratum spinosum, or stratum basale/germinativum, can have a thickness of at most about 50 mm, 40 mm, 20 mm, 10 mm, 8 mm, 4 mm, 2 mm, 1 mm, 0.8 mm, 0.4 mm, 0.2 mm, 0.15 mm, 0.1 mm, 0.08 mm, 0.04 mm, 0.02 mm, or 0.01 mm.

An epidermal layer can further comprise cells producing pigments, e.g., melanin. Such pigment-producing cells can be melanocytes. Melanocytes in the epidermal layer can express one or more markers. Such markers can include, but are not limited to, SRY-box containing gene 10 (Sox-10), Microphthalmia-associated transcription factor (MITF-M), premelanosome protein (gp-100), Dopachrome tautomerase (DCT), Tyrosinase (TYR), and Melan-A (MLANA).

### Cells in Synthetic Leather

A synthetic leather can comprise cells in the dermal layer and epidermal layer disclosed herein. In some embodiments, a synthetic leather may not comprise cells in the dermal layer. In some embodiments, a synthetic leather may not comprise cells in the epidermal layer disclosed. In some cases, a synthetic leather also can comprise hair follicle cells, endothelial cells, dermal papilla cells, immune system cells (such as lymphocytes, dendritic cells, macrophages or Langerhans cells), adipocytes, nerve cells, and a mixture thereof.

A layered structure containing, for example, an artificial dermal layer and a hair follicle, can be used to generate artificial fur. Such artificial fur can be engineered to mimic a look, color pattern, or fragrance of a natural animal. In some cases, a look, color pattern, or fragrance may be substantially different than a natural animal. In some cases, a dermal layer comprising cells of a particular animal can be used to generate an artificial fur of the same animal. In some cases, a dermal layer comprising cells of a particular animal can be used to generate an artificial fur of a different animal.

One or more cells in a synthetic leather can be genetically engineered cells. The term “genetically engineered” can refer to a man-made alteration to the nucleic acid content of a cell. Therefore, genetically engineered cells can include cells containing an insertion, deletion, and/or substitution of one or more nucleotides in the genome of a cell as well as alterations including the introduction of self-replicating extrachromosomal nucleic acids inserted into the cell. Genetically engineered cells also include those in which transcription of one or more genes has been altered, e.g., increased or reduced.

In some cases, a synthetic leather has at least one of the components of native skin such as melanocytes, hair follicles, sweat glands and/or nerve endings. In certain cases, a synthetic leather can be distinguished from normal native skin by its lack of at least one of these components. In some cases, displaying abnormal phenotypes or having at least one cell with an altered genotype, a synthetic leather can include all of these components.

In some case, additional components can be added to a synthetic leather. Such additional components can include myoepithelial cells, duct cells, secretory cells, alveolar cells, langerhans cells, Merkel cells, adhesions, mammary glands, or any mixture thereof. In some cases, a synthetic leather can comprise one or more of: neural cells, connective tissue (including bone, cartilage, cells differentiating into bone forming cells and chondrocytes, and lymph tissues), epithelial cells (including endothelial cells that form linings in cavities and vessels or channels, exocrine secretory epithelial cells, epithelial absorptive cells, keratinizing epithelial cells, and extracellular matrix secretion cells), or undifferentiated cells (such as embryonic cells, stem cells, or other precursor cells).

A synthetic leather can comprise hair follicles. A hair follicle can comprise one or more structures, including papilla, matrix, root sheath, bulge, infundibulum, the arrector pili muscles, the sebaceous glands, and the apocrine sweat glands. A hair follicle can comprise one or more hair follicle cells, including dermal papilla cell, outer root sheath cell, or any combination thereof. In some cases, a hair follicle can be in an epidermal layer. In some cases, a hair follicle can be in a dermal layer. A hair follicles cell can be differentiated from a progenitor, e.g., a stem cell such as an iPSC. In some embodiments, at least about 1%, 2%, 3%, 4%, 5%, 6%, 7%, 8%, 9%, 10%, 20%, 30%, 40%, 50%, 60%, 70%, 80%, 90%, 100% of hair follicle cells can be differentiated from induced pluripotent stem cells.

In some embodiments, a synthetic leather can be devoid of hair, blood vessels, sebaceous glands, hair follicle, oil glands, nerve, or a combination thereof In some cases, a synthetic leather can comprise hairs, e.g., in one or more layered structures. For example, a synthetic leather can comprise fur. The hairs (e.g., fur) can be natural, synthetic, or a combination thereof. The hairs (e.g., fur) can be grown from cells in the synthetic leather or added to synthetic leather from an exogenous source. In other cases, a synthetic leather may not have any hairs.

One or more cells in a synthetic leather can be differentiated from progenitor cells, such as stem cells. For example, fibroblasts in a synthetic leather can be differentiated from stem cells. For example, keratinocytes in a synthetic leather can be differentiated from stem cells. For example, melanocytes in a synthetic leather can be differentiated from stem cells. In some embodiments, at least about 1%, 2%, 3%, 4%, 5%, 6%, 7%, 8%, 9%, 10%, 20%, 30%, 40%, 50%, 60%, 70%, 80%, 90%, 100% of cells disclosed herein can be differentiated from stem cells. In some embodiments, at least about 1%, 2%, 3%, 4%, 5%, 6%, 7%, 8%, 9%, 10%, 20%, 30%, 40%, 50%, 60%, 70%, 80%, 90%, 100% of fibroblasts can be differentiated from induced pluripotent stem cells. In some embodiments, at least about 1%, 2%, 3%, 4%, 5%, 6%, 7%, 8%, 9%, 10%, 20%, 30%, 40%, 50%, 60%, 70%, 80%, 90%, 100% of keratinocytes can be differentiated from induced pluripotent stem cells. In some embodiments, at least about 1%, 2%, 3%, 4%, 5%, 6%, 7%, 8%, 9%, 10%, 20%, 30%, 40%, 50%, 60%, 70%, 80%, 90%, 100% of melanocytes cells can be differentiated from induced pluripotent stem cells. In some cases, cells can be derived from adipose, umbilical cord stem cells, stem cells isolated from milk, mesenchymal stem cells, or any combination thereof.

Stem cells can be embryonic stem cells (ESCs), adult stem cells (i.e., somatic stem cells) or induced pluripotent stem cells (iPSCs). In some embodiments, a stem cell can be totipotent, pluripotent or multipotent for example adult stem cells and cord blood stem cells). Embryonic stem cells can be derived from fertilized embryos that are less than one week old. Induced pluripotent stem cells can be obtained through the induced expression of one or more of Oct3, Oct4, Sox2, Klf4, and c-Myc genes in any somatic cell (e.g., adult somatic cell) such as fibroblast. In some cases, one or more other genes can also be induced for reprograming a somatic cell to an induced pluripotent stem cell. Examples of such genes include NANOG, UTF1, LIN28, SALL4, NR5A2, TBX3, ESSRB, DPPA4, SV40LT, REM2, MDM2, and cyclin D1.

Various delivery methods can be used to modulate the expression of genes to reprogram a somatic cell to an iPSC. Exemplary delivery methods include naked DNA delivery, adenovirus, electrical delivery, chemical delivery, mechanical delivery, polymer-based systems, microinjection, retroviruses (e.g., MMLV-derived retroviruses), and lentiviruses (e.g., excisable lentiviruses). In some cases, induced pluripotent stem cells can be obtained according to a protocol known in the art. In some case, somatic cells (e.g., adult somatic cells) are transfected with viral vectors, such as retroviral vectors, which comprise Oct3, Oct4, Sox2, Klf4, and c-Myc genes. In some cases, Sendai viruses are used as a delivery system, e.g., Sendai viruses produced by ID Pharma Co., Ltd., Japan.

### Sources of Cells

A synthetic leather can comprise cells derived from animals of one or more species. For example, the cells in a synthetic leather can be derived from mammals, birds, reptiles, amphibian, fish, invertebrates, or any combination thereof.

A synthetic leather can comprise cells derived from mammals, e.g., mammalian cells. A mammal can be a non-human mammal. A non-human mammal can be antelope, bear, beaver, bison, boar, camel, caribou, cat, cattle, deer, dog, elephant, elk, fox, giraffe, goat, hare, horse, ibex, kangaroo, lion, llama, lynx, mink, moose, oxen, peccary, pig, rabbit, rhino, seal, sheep, squirrel, tiger, whale, wolf, yak, or zebra. In some cases, a mammal can be primate, bovine, ovine, porcine, equinine, canine, feline, rodent, lagomorph, fish, bird or a reptile. In some cases, a mammal can be a human. In some embodiments a human can be a celebrity. As used herein, the term “celebrity” can be defined as a person that has come into the community attention by way of notoriety or general fame of previous activities. A “celebrity” can be associated with industries including but not limited to professional and amateur sports, entertainment, music, motion picture, business, print and electronic media, politics, and the like.

A synthetic leather can comprise cells derived from other species. In some cases, the cells are derived from birds, such as chicken, duck, emu, goose, grouse, ostrich, pheasant, pigeon, quail, or turkey. In some cases, the cells are derived from reptiles such as turtle, snake, crocodile, or alligator. In some cases, the cells are derived from amphibians such as frog, toad, salamander, or newt. In some cases, the cells are derived from fish, such as anchovy, bass, catfish, carp, cod, eel, flounder, fugu, grouper, haddock, halibut, herring, mackerel, mahi-mahi, manta ray, marlin, orange roughy, perch, pike, pollock, salmon, sardine, shark, snapper, sole, stingray, swordfish, tilapia, trout, tuna, or walleye.

In some cases, all cells in a synthetic leather are derived from the same species. For example, all cells in a synthetic leather can be bovine cells. In other cases, a synthetic leather can comprise cells derived from multiple species. For example, a synthetic leather can comprise bovine cells and alligator cells. In some cases, a synthetic leather can comprise cells derived from at least 2, 3, 4, 5, 6, 7, 8, or 10 species.

Progenitors of the cells in a synthetic leather can also be derived from the sources described herein. For example, stem cells (e.g., iPSCs), somatic cells (e.g., to be reprogramed to iPSCs), primary cells used in synthetic cells, dermal layer cells, epidermal layer cells, or any cells in the synthetic and their progenitors thereof can be derived from the sources described herein. In some cases, primary cells can be used. In some cases, fibroblast cells can be used. In some cases, primary bovine fibroblast cells can be used. In some cases, cells can be derived from stem cells. In some cases, cells can be derived from immortalized cells.

In some cases, an immortalized cell line can be used. An “immortalized cell line” as used herein can refer to a population of cells that normally do not proliferate, but have acquired an ability to do so indefinitely, for example, through mutation. Examples of immortalized cells can include CHO, BHK, HEK 293A, HEK 293T, HeLa, 3T3, A549, Jurkat, Vero, BT-20, EvsaY, MCF-7, SkBr3, T-47D, IMR-90, NRK49F, BRL 3A, Sk/HEP-1, Caco-2, Friend, and MCF-7 cells. In some cases, an immortalized cell line can grow in suspension without being attached to a microcarrier or being reliant on cell/cell contact (cell aggregates). In some embodiments, a cell can be switched back to a non-proliferating cell once seeded and expanded on a 3D surface. In some embodiments, a cell line can be designed to produce collagen and other ECM proteins with minimal growth factor input.

Any cell can be a live cell or a dead cell. When multiple cells are present, a cell may be a live cell, may be a dead cell, or any combination thereof.

### Layered Structure

A synthetic leather can comprise one or more layered structures. In some embodiments, a layered structure or parts thereof can be cultured or grown in a system described herein. A layered structure can be formed by placing a first type of layer upon a second type of layer. The first type of layer and the second type of layer can be the same or different. In some cases, a layered structure can be formed by placing an epidermal layer upon a dermal layer. For example, a layered structure can be formed by placing an epidermal layer upon a dermal layer, with a basement membrane substitute in between. In some embodiments, a layered structure or a portion thereof can be grown/cultured in a container disclosure herein.

A layered structure can comprise two or more layers. In some cases, a layered structure can comprise at least 2, 3, 4, 5 6, 7, 8, 9, 10, 15, 20, 30, 40, 50, 60, 70, 80, 90, 100, 500, or 1000 layers. In some cases, a layered structure can comprise at least 2, 3, 4, 5 6, 7, 8, 9, 10, 15, 20, 30, 40, 50, 60, 70, 80, 90, 100, 500, or 1000 first type of layers, and at least 2, 3, 4, 5 6, 7, 8, 9, 10, 15, 20, 30, 40, or 50 second type of layers. For example, a layered structure can comprise at least 2, 3, 4, 5 6, 7, 8, 9, 10, 15, 20, 30, 40, 50, 60, 70, 80, 90, 100, 500, or 1000 dermal layers, and at least 2, 3, 4, 5 6, 7, 8, 9, 10, 15, 20, 30, 40, 50, 60, 70, 80, 90, 100, 500, or 1000 layers of epidermal layers.

A layered structure can comprise one or more types of cells described herein. For example, a layered structure can comprise cells in a dermal layer, such as fibroblasts, cells in an epidermal layer, such as keratinocytes, or any combination thereof. In some cases, a layered structure further can comprise cells other than fibroblasts and keratinocytes. For example, a layered structure can comprise melanocytes.

A layered structure can have a thickness from about 0.001 mm to about 100 mm. For example, a layered structure can have a thickness from about 0.005 mm to about 50 mm, from about 0.005 to about 10, from about 0.01 mm to about 10 mm, from about 0.02 to about 5 mm, from about 0.05 to about 5 mm, from about 0.1 to about 5 mm, from about 0.1 to about 2 mm, from about 0.1 to about 1 mm, or from about 0.1 to about 0.5 mm. In some cases, the thickness of a layered structure can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, 10 mm, 20 mm, 40 mm, 60 mm, 80 mm, or 100 mm. In some cases, the thickness of a layered structure can be at most 100 mm, 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a layered structure can have a thickness of at least about 100, 200, 300, 400, 500, 600, 700, 800 mm.

The length of a layered structure can be engineered to fit the function or use of a synthetic leather. A layered structure can have a length from about 0.01 mm to about 50 m. For example, a layered structure can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a layered structure can have a length from about 0.02 mm to 5 mm. For example, a layered structure can have a length from about 0.1 mm to 0.5 mm. For example, a layered structure can have a length from about 0.2 mm to 0.5 mm. In some cases, the length of a layered structure can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the length of a layered structure can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a layered structure can have a length of at least about 50, 60, 70, 80, 90, 100, 200, 300, 400, 500, 700, 1000 mm. In some embodiments, a layered structure can have a length of at least about 50, 60, 70, 80, 90, 100, 200, 300, 400, 500, 600, 700 cm. In some embodiments, a layered structure can have a length of at least about 50, 60, 70, 80, 90, 100, 200, 300, 400 m.

The width of a layered structure can be engineered to fit the function or use of a synthetic leather. A layered structure can have a width from about 0.01 mm to about 50 m. For example, a layered structure can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a layered structure can have a width from about 0.02 mm to 5 mm. For example, a layered structure can have a width from about 0.1 mm to 0.5 mm. For example, a layered structure can have a width from about 0.2 mm to 0.5 mm. In some cases, the width of a layered structure can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the width of a layered structure can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a layered structure can have a width of at least about 50, 60, 70, 80, 90, 100, 200, 300, 400, 500, 700, 1000 mm. In some embodiments, a layered structure can have a width of at least about 50, 60, 70, 80, 90, 100, 200, 300, 400, 500, 600, 700 cm. In some embodiments, a layered structure can have a width of at least about 50, 60, 70, 80, 90, 100, 200, 300, 400 m.

A layered structure can comprise fibroblasts and keratinocytes at any ratio of at least about 50:1, 40:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:10, or 1:100. In some cases, the ratio of fibroblasts to keratinocytes can be from about 20:1 to about 3:1, from about 20:1 to about 4:1, from about 20:1 to about 5:1, from about 20:1 to about 10:1, or from about 20:1 to about 15:1.

A layered structure can comprise fibroblasts and melanocytes at any ratio of at least about 50:1, 40:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:10, or 1:100. In some cases, the ratio of fibroblasts to melanocyte can be from about 20:1 to about 3:1, from about 20:1 to about 4:1, from about 20:1 to about 5:1, from about 20:1 to about 10:1, or from about 20:1 to about 15:1.

A layered structure can comprise keratinocytes and melanocytes at any ratio of at least about 50:1, 40:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:10, or 1:100. In some cases, the ratio of keratinocytes to melanocyte can be from about 20:1 to about 3:1, from about 20:1 to about 4:1, from about 20:1 to about 5:1, from about 20:1 to about 10:1, or from about 20:1 to about 15:1.

One type of cells in a layered structure can comprise at most 99%, 95%, 90%, 85%, 80%, 75%, 70%, 65%, 60%, 55%, 50%, 45%, 40%, 35%, 30%, 25%, 20%, 10%, 5%, or 1% of the total cell population in the layered structure. One type of cells in a layered structure can comprise about at least 1%, 5%, 10%, 20%, 30%, 40%, 50%, 60%, 65%, 70%, 75%, 80%, 85%, 90%, or 95% of the total cell population in the layered structure. For example, fibroblasts in a layered structure can comprise about at least 5%, 10%, 20%, 30%, 40%, 50%, 60%, 65%, 70%, 75%, 80%, 85%, 90%, or 95% of the total cell population in the layered structure.

### Synthetic Leather

A synthetic leather can be formed by one or more layered structures. For example, a synthetic leather can be formed by at least 1, 2, 3, 4, 5, 6, 7, 8, 9, 10, 15, 20, 30, 40, 50, 60, 70, 80, 90, or 100 layered structures.

A synthetic leather can be of various thickness. For example, a synthetic leather can have a thickness resembling to a natural leather. In some cases, a synthetic leather can have a thickness from about 0.001 mm to about 100 mm. For example, a layered structure can have a thickness from about 0.005 mm to about 50 mm, from about 0.005 to about 10, from about 0.01 mm to about 10 mm, from about 0.1 to about 5 mm, from about 0.5 mm to about 5 mm, from about 0.5 mm to about 3 mm, from about 0.8 mm to about 3 mm, from about 0.8 mm to about 2 mm, from about 0.8 mm to about 1.8 mm, from about 0.8 mm to about 1.6 mm, from about 0.9 mm to about 1.4 mm, from about 1 mm to about 1.5 mm, from about 1 mm to about 1.4 mm, or from about 1 mm to about 1.3 mm. In some cases, the thickness of a synthetic leather can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, 10 mm, 20 mm, 40 mm, 60 mm, 80 mm, or 100 mm. In some cases, the thickness of a synthetic leather can be at most 100 mm, 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some cases, the thickness of a synthetic leather can be about 1.2 mm.

A synthetic leather can have a length from about 0.01 mm to about 50 m. For example, a synthetic leather can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a synthetic leather can have a length from about 0.02 mm to 5 mm. For example, a synthetic leather can have a length from about 0.1 mm to 0.5 mm. For example, a synthetic leather can have a length from about 0.2 mm to 0.5 mm. In some cases, the length of a synthetic leather can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the length of a synthetic leather can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a synthetic leather can have a length of at least about 50, 60, 70, 80, 90, 100, 200, 300, 400, 500, 700, 1000 mm. In some embodiments, a synthetic leather can have a length of at least about 50, 60, 70, 80, 90, 100, 200, 300, 400, 500, 600, 700 cm. In some embodiments, a synthetic leather can have a length of at least about 50, 60, 70, 80, 90, 100, 200, 300, 400 m.

A synthetic leather can have a width from about 0.01 mm to about 50 m. For example, a synthetic leather can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. For example, a synthetic leather can have a width from about 0.02 mm to 5 mm. For example, a synthetic leather can have a width from about 0.1 mm to 0.5 mm. For example, a synthetic leather can have a width from about 0.2 mm to 0.5 mm. In some cases, the width of a synthetic leather can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some cases, the width of a synthetic leather can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a synthetic leather can have a width of at least about 50, 60, 70, 80, 90, 100, 200, 300, 400, 500, 700, 1000 mm. In some embodiments, a synthetic leather can have a width of at least about 50, 60, 70, 80, 90, 100, 200, 300, 400, 500, 600, 700 cm. In some embodiments, a synthetic leather can have a width of at least about 50, 60, 70, 80, 90, 100, 200, 300, 400 m. In some embodiments, synthetic leather or a portion thereof can be grown/cultured in a container disclosure herein.

### Basement Membrane Substitute

A synthetic leather can further comprise a basement membrane substitute. In some embodiments, a basement membrane substitute or parts thereof can be cultured or grown in a system described herein. A basement membrane substitute can be between two cell layers, e.g., between a dermal layer and an epidermal layer. A basement membrane substitute can be a dermo-epidermal junction similar to that which exists in vivo, from a structural point of view and/or from a biochemical point of view. From the biochemical point of view, a basement membrane substitute can comprise components of the basal membrane, of the lamina densa, of the lamina lucida and of the sub-basal zone, such as, collagen IV, collagen VII, laminin 5, entactin fibronectin, or any combination thereof.

In some embodiments, a basement membrane substitute in a synthetic leather can comprise a urinary basement membrane (UBM), a liver basement membrane (LBM), an amnion, a chorion, an allograft pericardium, an allograft acellular dermis, an amniotic membrane, a Wharton's jelly, a vitronectin, a fibronectin, a laminin, a protein mixture, or any combination thereof. In some embodiments, a protein mixture can be secreted by a cell. In some embodiments, a protein mixture can be secreted by Engelbreth-Holm-Swarm (EHS) mouse sarcoma cells. In some embodiments, a basement membrane substitute can be a dried acellular amniotic membrane. In certain cases, a basement membrane substitute can be a polymer, e.g., a nanopolymer. For example, a basement membrane substitute can be nano-fibrous poly hydroxybutyrate-cohydroxyvalerate (PHBV). In some embodiments, a basement membrane substitute or parts thereof can be grown/cultured in a container disclosure herein.

### Scaffold

In some embodiments, scaffolds can comprise a fundamental component of tissue generation, reparative, restorative and regenerative strategies, and development of advanced scaffolds can be crucial to successful tissue engineering. In some embodiments, cells can be grown/cultured on scaffolds disclosed herein in a container disclosure herein.

In some embodiments, a scaffold as disclosed herein can be produced in a container of a modular, scalable bioreactor as described herein. In some embodiments, a scaffold disclosed herein can be manufactured for in vivo, in vitro, or ex vivo use. In some embodiments, a scaffold disclosed herein can be for the growth and manufacture of synthetic leathers, artificial epidermal layers, artificial dermal layers, layered structures, products made thereof and methods of producing the same. In some embodiments, full thickness skin equivalent can comprise at least one dermal layer and at least one epidermal layer. In some embodiments, full thickness skin equivalent and full skin equivalent can be used interchangeably. In some embodiments, a scaffold disclosed herein can comprise a layer of artificial dermal layer comprising a fibroblast and an artificial epidermal layer comprising a keratinocyte. In some embodiments, a dermal layer and an epidermal layer can form a layered structure. In some embodiments, a synthetic leather can comprise one or more layered structure. In some embodiments, a synthetic leather can be tanned and further processed. In some embodiments, cells forming a synthetic layer can be differentiated from stem cells, e.g., induced pluripotent stem cells (iPSC). In some embodiments, a dermal layer can be placed on a scaffold disclosed herein.

In some embodiments, scaffolds as described herein may be any shape suitable for a particular in vitro or in vivo application. In some embodiments, a scaffold can be a conduit. In some embodiments, a conduit can have at least two openings and a passageway connecting the openings. In some embodiments, an exterior of a conduit may possess any shape suitable for an application. For example, an exterior of a conduit may be tubular in shape. In some embodiments, a wall of a passageway of a conduit (i.e., the interior) also may possess any shape suitable for an application. Thus, cross-sections taken at different locations along a length of a conduit may have differing areas, revealing an irregularly shaped interior. In some embodiments, a cross-section can be round, elliptical, or irregularly polygonal, depending on an application. In some embodiments, a scaffold as described herein can be used for skin, bone, cartilage, and/or soft tissue repair. In some embodiments, a scaffold as described herein can be used in virtually all instances when it is desirable to provide a substrate for the growth of cells onto or into a tissue replaceable matrix, either in vitro or in vivo.

### Delivery Vehicle

Disclosed herein in some embodiments, are compositions that can be used as a vehicle for an in-situ delivery of biologically active agents. In some embodiments, a biologically active agent can be incorporated into, or included as an additive within, a composition as described herein. In some embodiments, a biologically active agent can comprise a medicament, a vitamin; a mineral supplement; a substance used for a treatment, a prevention, a diagnosis, a cure or a mitigation of a disease or an illness; a substance which can affect a structure or a function of a body; a drug, or any combination thereof. In some embodiments, a biologically active agent can be used, to facilitate implantation of a composition into a subject. In some embodiments, a biologically active agent can be used to promoteintegration and healing processes. In some embodiments, a biologically active agent can be used, to facilitate growth and/or development of cells placed on a scaffold. In some embodiments, a biologically active agent can comprise an antibody, an antibody fragment, an antibiotic, an antifungal agent, an antibacterial agent, an anti-viral agent, an anti-parasitic agent, a growth factor, a neurotrophic factor, an angiogenic factor, an anaesthetic, a mucopolysaccharide, a metal, a cell, a protein, a polynucleotide, a polypeptide enzyme, a degradation agent, a lipid, a carbohydrate, a chemical compound such as pharmaceuticals and other wound healing agents, or any combination thereof. In some embodiments, a biologically active agent can be a therapeutic agent, a diagnostic material, a research reagent, or any combination thereof.

In some embodiments, a cell graphed onto or placed on a scaffold described herein can be stimulated, inhibited or caused to differentiate or dedifferentiate by contact with one or more differentiation agents. In some embodiments, a differentiation agent can comprise a trophic factor, a hormonal supplement, a forskolin, a retinoic acid, a putrescin-transferrin, a cholera toxin, an insulin-like growth factor (IGF), a transforming growth factor (e.g., TGF-α, TGF-β), a tumor necrosis factor (TNF), a fibroblast growth factor (FGF), an epidermal growth factor (EGF), a granulocyte macrophage-colony stimulating factor (GM-CSF), a hepatocyte growth factor (HGF), a hedgehog, a vascular endothelial growth factor (VEGF), a thyrotropin releasing hormone (TRH), a platelet derived growth factor (PDGF), a sodium butyrate, a butyric acid, a cyclic adenosine monophosphate (cAMP), a cAMP derivatives (e.g., dibutyryl cAMP, 8-bromo-cAMP), a phosphodiesterase inhibitors, an adenylate cyclase activators, a prostaglandins, a ciliary neurotrophic factor (CNTF), a brain-derived neurotrophic factor (BDNF), a neurotrophin 3, a neurotrophin 4, an interleukins (e.g., IL-4), an interferons (e.g., interferon-gamma), a potassium, a amphiregulin, a dexamethasone (glucocorticoid hormone), an isobutyl 3-methyulxanthine, a somatostatin, a lithium, a growth hormone, or any combination thereof.

### Scaffold Application

As used herein, the term “scaffold” can refer to a highly porous, artificial, three-dimensional network of interconnected pores that is used in vivo, in vitro, or ex vivo as a framework to which additional cells can attach and both existing and additional cells can grow. In some embodiments, a scaffold can be formed from or by living cells. In other embodiments, one or more species of living cells can be attached to a scaffold disclosed herein via physical bonding or chemical bonding described herein. The living cells can be allowed to proliferate for a time period, in which the cells can grow to form colonies, after which the colonies can fuse to form a network of cells and subsequently forming a living scaffold.

In some embodiments, a scaffold can be used for a wide variety of applications, e.g. tissue engineering. In some embodiments, a three-dimensional expansion of autologous cells like bone marrow mesenchymal stem cells which are limited due to donor site morbidity. In some embodiments, a host for such applications can be an animal. In some embodiments, a host can be a mammal or a human patient.

In some embodiments, a scaffold can be used in transplantation as a matrix, for dissociated cells to create a three-dimensional tissue or organ. In some embodiments, dissociated cells can comprise chondrocytes or hepatocytes. In some embodiments, a type of cell can be added to a scaffold for culturing and possible implantation, including cells of a muscular and skeletal system, such as a chondrocyte, a fibroblast, a muscle cell, an osteocyte, a parenchymal cell, a hepatocyte, a pancreatic cell (including Islet cells), a cell of intestinal origin, a nerve cell, a skin cell, or any combination thereof. In some embodiments, a cell can be obtained from a donor, from an established cell culture line, or a combination thereof. In some embodiments, a cell can be obtained before or after genetic engineering. In some embodiments, a piece of tissue can be used, which can provide a number of different cell types in the same structure. In some embodiments, a scaffold can be used as a three-dimensional in vitro culture system for attachment-dependent cells, e.g., hepatocytes in a three-dimensional microenvironment, which mimics the physiological microenvironment more closely.

In some embodiments, a scaffold can be used to repair a damaged tissue. Disclosed herein in some embodiments, is a method of repairing a damaged tissue. In some embodiments, a method of repairing a damaged tissue can comprise providing a scaffold obtainable or obtained by a method described herein and contacting a scaffold with a damaged tissue of a subject in need thereof. In some embodiments, skin cells, for example, fibroblast, keratinocytes, melanocytes, Langerhans cells, or Merkel cells can be seeded onto a scaffold described herein. In some embodiments, one or more of embryonic stem cells, adult stem cells, blast cells, cloned cells, fertilized ova, placental cells, keratinocytes, basal epidermal cells, hair shaft cells, hair-root sheath cells, surface epithelial cells, basal epithelial cells, urinary epithelial cells, salivary gland cells, mucous cells, serous cells, von Ebner's gland cells, mammary gland cells, lacrimal gland cells, ceruminous gland cells, eccrine sweat gland cells, apocrine sweat gland cells, Moll gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing β cells, glucagon-producing a cells, somatostatin-producing δ cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peripolar cells of the kidney, mesangial cells of the kidney, brush border cells of the intestine, striated duct cells of exocrine glands, gall bladder epithelial cells, brush border cells of the proximal tubule of the kidney, distal tubule cells of the kidney, nonciliated cells of ductulus efferens, epididymal principal cells, epididymal basal cells, hepatacytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroids plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, immunoglobulin M, immunoglobulin G, immunoglobulin A, immunoglobulin E, killer cells, rod cells, cone cells, inner hair cells of the organ of Corti, outer hair cells of the organ of Corti, type I hair cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialized for touch, primary sensory neurons specialized for temperature, primary neurons specialized for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian follicle cells, Sertoli cells, and thymus epithelial cells, or combinations thereof may be seeded onto a scaffold disclosed herein.

A cell layer (e.g., a dermal layer), a layered structure, or a synthetic leather can be placed on a scaffold. A scaffold can provide certain firmness (e.g., resistance to tearing), elasticity, or both. In some cases, a part of or the entire scaffold can be comprised in a product made from cells grown on a scaffold described herein. In other cases, a scaffold is not comprised in a product made from cells grown on a scaffold described herein. After assisting the formation of tissue, an organ or a layer of cells, a scaffold can be removed. In certain cases, a scaffold can be degraded after a period of time.

### Scaffold Composition

A scaffold described herein can be made of natural materials, synthetic materials, or combination thereof. Examples of scaffolds include a scaffold formed using a net made of a bioabsorbable synthetic polymer, a scaffold formed by attaching a nylon net to a silicon film, a scaffold having a two-layered structure of a collagen sponge and a silicon sheet, a scaffold formed using an atelo collagen sponge made into a sheet, a scaffold formed by matching collagen sponges having different pore sizes, and acellular dermal matrices (ADM) formed using fibrin glue or allogeneic skin that has been made cell-free.

A scaffold can comprise natural substances such as collagen (e.g., collagen matrix), natural adhesive (e.g., fibrin glue, cold glues, animal glue, blood albumen glue, casein glue, or vegetable glues such as starch and dextrin glues). In some cases, a scaffold can comprise silk. For example, a scaffold can be made of silk. In some embodiments, a scaffold can comprise, silk fibroin, cellulose, cotton, acetate, acrylic, latex fibers, linen, nylon, rayon, velvet, modacrylic, olefin polyester, saran, vinyon, wool, jute, hemp, bamboo, flax or a combination thereof. In some embodiments, a scaffold can comprise fibers. In some embodiments, the fibers can be fibers of silk, cotton, wool, linen, cellulose extracted in particular from wood, vegetables or algae, polyamide, modified cellulose (rayon, viscose, acetate, especially rayon acetate), poly-p-phenyleneterephthalamide, acrylic fibers, for example those of polymethyl methacrylate or of poly-2-hydroxyethyl methacrylate, fibers of polyolefin for example fibers of polyethylene or polypropylene, glass, silica, aramid, carbon, for example in the form of graphite, poly(tetrafluoroethylene), insoluble collagen, polyesters, polyvinyl chloride or polyvinylidene chloride, polyvinyl alcohol, polyacrylonitrile, chitosan, polyurethane, poly(urethane-urea) or polyethylene phthalate, and fibers formed from a blend of polymers such as those mentioned above, such as polyamide/polyester fibers or any combination thereof.

A scaffold can comprise polymers. A polymer can be a biopolymer. A biopolymer can include but is not limited to chitin, chitosan, elastin, collagen, keratin or polyhydroxyalkanoate. The polymers can be biodegradable, biostable, or combinations thereof. The polymer in a scaffold can be natural polymers. Exemplary natural polymers include polysaccharides such as alginate, cellulose, dextran, pullane, polyhyaluronic acid, chitin, poly(3-hydroxyalkanoate), poly(3-hydroxyoctanoate) or poly(3-hydroxyfatty acid). In some cases, a scaffold also can comprise chemical derivatives of the natural polymers. Such chemical derivatives can include substitutions and/or additions of chemical groups such as alkyl, alkylene, hydroxylations, oxidations, as well as other modifications familiar to those skilled in the art. The natural polymers can also be selected from proteins such as collagen, zein, casein, gelatin, gluten, and serum albumen. The polymer in a scaffold can be biodegradable synthetic polymers, including poly alpha-hydroxy acids such as poly L-lactic acid (PLA), polyglycolic acid (PGA) or copolymers thereof (e.g., poly D,L-lactic co-glycolic acid (PLGA)), and hyaluronic acid.

A scaffold can be bioabsorbable. A bioabsorbable scaffold is a non-cytotoxic structure or substance that is capable of containing or supporting living cells and holding them in a desired configuration for a period of time. The term “bioabsorbable” can refer to any material the body can break down into non-toxic by-products that are excreted from the body or metabolized therein. Exemplary bioabsorbable materials for a scaffold include, poly(lactic acid), poly(glycolic acid), poly(trimethylene carbonate), poly(dimethyltrimethylene carbonate), poly(amino acids)s, tyrosine-derived poly(carbonates)s, poly(carbonates)s, poly(caprolactone), poly(para-dioxanone), poly(esters)s, poly(ester-amides)s, poly(anhydrides)s, poly(ortho esters)s, collagen, gelatin, serum albumin, proteins, polysaccharides, mucopolysaccharides, carbohydrates, glycosaminoglycans, poly(ethylene glycols)s, poly(propylene glycols)s, poly(acrylate esters)s, poly(methacrylate esters)s, poly(vinyl alcohol), hyaluronic acid, chondroitin sulfate, heparin, dermatan sulfate, versican, copolymers, blends and mixtures of polymers, and oligomers containing bioabsorbable linkages.

A scaffold can comprise, polyethylenes, polyvinyl chlorides, polyamides such as nylons, polyesters, rayons, polypropylenes, polyacrylonitriles, acrylics, polyisoprenes, polybutadienes and polybutadiene-polyisoprene copolymers, neoprenes and nitrile rubbers, polyisobutylenes, olefinic rubbers such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates such as ethylene vinyl acetate copolymer, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art. Other biocompatible nondegradable polymers that are useful in accordance with the present disclosure include polymers comprising biocompatible metal ions or ionic coatings which can interact with DNA. Such metal ions include, but are not limited to gold and silver ions, Al³⁺, Fe³⁺, Fe²⁺, Mg²⁺, and Mn²⁺. In exemplary embodiments, gold and silver ions may be used, for example, for inhibiting inflammation, binding DNA, and inhibiting infection and thrombosis.

A scaffold disclosed herein can comprise poly(lactide) (PLA), poly(glycolic acid) (PGA), poly(lactide-co-glycolide) (PLGA), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, teflon™, or nylon.

A scaffold can comprise polymers and oligomers of glycolide, lactide, polylactic acid, polyesters of a-hydroxy acids, including lactic acid and glycolic acid, such as the poly(a-hydroxy) acids including polyglycolic acid, poly-DL-lactic, poly-L-lactic acid, and terpolymers of DL-lactide and glycolide; e-caprolactone and e-caprolactone copolymerized with polyesters; polylactones and polycaprolactones including poly(e-caprolactone), poly(8-valerolactone) and poly(gamma-butyrolactone); polyanhydrides; polyorthoesters; other hydroxy acids; polydioxanone; and other biologically degradable polymers that are non-toxic or are present as metabolites in the body. Examples of polyaminoacids include, but are not limited to, polylysine (PLL), poly L-aspartic acid, poly L-glutamic acid, and styrene-maleic acid anhydride copolymer. Examples of derivatives of polyethylene glycol includes, but are not limited to, poly(ethylene glycol)-di-(ethylphosphatidyl(ethylene glycol)) (PEDGA), poly(ethylene glycol)-co-anhydride, poly(ethylene glycol)co-lactide, poly(ethylene glycol)-co-glycolide and poly(ethylene glycol)-co-orthoester. Examples of acrylamide polymers include, but are not limited to, polyisopropylacrylamide, and polyacrylamide. Examples of acrylate polymers include, but are not limited to, diacrylates such as polyethylene glycol diacrylate (PEGDA), oligoacrylates, methacrylates, dimethacrylates, oligomethoacrylates and PEG-oligoglycolylacrylates. Examples of carboxy alkyl cellulose include, but are not limited to, carboxymethyl cellulose and partially oxidized cellulose. In one embodiment a polyethylene glycol diacrylate (PEGDA) has been used as hydrogel in the composition of the present disclosure.

A scaffold disclosed herein can comprise a polysaccharide. A polysaccharide can be chitosan, oligochitosan. Preferably, the carbodiimide is selected from N-ethyl-N′-(3-dimethylaminopropyl)-carbodiimide, NN′-dicyclohexyl-carbodiimide (DCC), N′-diisopropyl-carbodiimide, N′N′-di-tert-butylcarbodiimide 1-cyclo-hexyl-3-(4-diethylaminocyclohexyl)carbodiimide, 1,3-di-(4-diethylaminocyclo-hexyl)carbodiimide, 1-cyclohexyl-3-(-diethylaminoethylcarbodiimide, and 1-cyclohexyl-1-cyclohexyl-3-(2-morphonlinyl-(4)-ethyl)carbodiimide 1-cyclohexyl-3-(4-diethyl-aminocyclohexyl)carbodiimide. Also, because the scaffolds comprise potentially edible components, they are useful as the basis for synthetic meat products that grow and regenerate animal cells.

A scaffold composition can comprise a radical initiator. A radical initiator can be a photoinitiator. A photoinitiator can be a peroxide, a nitrogen dioxide, an azo compound, an acrylate, a phosphine oxide, and the like. In some cases, a photoinitiator can be benzoylperoxide; 2,2-dimethoxy-2-phenylacetophenone; polyethylene glycol diacrylate (PEGDA); trimethylolpropane triacrylate; acryloyl chloride; azobisisobutyronitrile; camphorquinone; 2-hydroxy-1-[4-(2-hydroxyethoxy) phenyl]-2-methyl-1-propanone; 2,4,6-trimethylbenzoyldiphenylphosphine oxide; or a salt of any of these. In some cases, a radical initiator is not a photoinitiator. In some cases, a radical initiator is water soluble. In some cases, a radical initiator is not water soluble.

In some embodiments, a scaffold can comprise components that can provide among other properties, mechanical properties, porosity, and increased surface area. Such components can include but not limited to hydrogel solutions, fibrinogen, thrombin, chitosan, collagen, alginate, poly(N-isopropylacrylamide), hyaluronate, polylactic acid (PLA), polyglycolic acid (PGA), and PLA-PGA co-polymers. In some embodiments for example, fibrinogen and thrombin can be co-deposited to provide a fibrin matrix. In other embodiments, fibrinogen may be cross-linked to growth factors.

In some embodiments, a scaffold disclosed herein can comprise a methacrylated chitosan material. In some embodiments, a silicate-based nanoparticle material (for example, laponite nanoparticles) can be combined with a sugar containing preparation, such as sucrose, in a defined volume of a physiologically acceptable buffer solution, such as Dulbecco's Phosphate Buffered Saline (DPBS), prior to being combined with a composite material for use in a scaffold disclosed herein.

In some embodiments, a scaffold composition can comprise two or more polymer or resins disclosed herein. In some aspects, a scaffold composition can comprise methacrylated chitosan and PEGDA resin. In some embodiments, a polymer, or resin can be combined at a ratio of 0.0005, 0.5, 1, 1.5, 2, 3, 4, 5, 6, 7, 8, 9, 10, 20, 30, 40, 50, 60, 70, 80, 90, 100, 200, 500, or at least about 1000.

### Scaffold Thickness

A scaffold can be of various thicknesses. For example, a scaffold can have a thickness that is suitable for forming a cell layer. For example, a scaffold can have a thickness from about 0.1 mm to about 10 mm, such as from about 0.1 mm to about 5 mm, from about 0.1 mm to about 4 mm, from about 0.1 mm to about 3 mm, from about 0.1 mm to about 2 mm, to about 0.1 mm to about 1 mm, from about 0.2 mm to about 1 mm, from about 0.3 mm to about 1 mm, from about 0.4 mm to about 1 mm, from about 0.5 mm to about 1 mm, from 0.3 mm to about 1.5 mm, from about 0.4 mm to about 1.2 mm, from about 0.6 mm to about 1.2 mm, or from about 0.7 mm to about 1.5 mm. For example, a scaffold can have a thickness from about 0.5 mm to 1 mm. In some cases, a scaffold can be at least 0.1 mm, 0.2 mm, 0.3 mm, 0.4 mm, 0.5 mm, 0.8 mm, 1 mm, 2 mm, 3 mm, 4 mm, or 5 mm thick. In some cases, a scaffold can be at most 0.5 mm, 0.8 mm, 1 mm, 2 mm, 3 mm, 4 mm, 5 mm, 6 mm, 7 mm, 8 mm, 9 mm, or 10 mm thick. In some embodiments, a scaffold can have a length and/or a width of a cell layer to be placed and/or grown upon a scaffold. In some embodiments, a scaffold can have a length and/or a width of a cell layer described herein.

### Scaffold Formation

Disclosed herein are methods, compositions and apparatus for creating a scaffold. In accordance with the disclosure, solid freeform fabrication (SFF) processes and apparatus can be used in a layering manufacturing process to build up shapes by incremental materials deposition and fusion of thin cross-sectional layers. In certain embodiments, a scaffold can be created ex vivo or in situ.

A scaffold can be utilized in the modular, scalable system described herein. In some embodiments, a scaffold can be fabricated in a container of a system described herein. In some embodiments, a scaffold can be fabricated separately and added to a container of a system described herein.

The 3-D structure of a scaffold may be fabricated directly using SFF. For example, magnetic resonance imaging (MRI) or computerized axial tomography (CAT) scans may be used to determine the 3-D shape of an in vivo structure which is to be repaired or replaced. Computer-aided-design (CAD) or computer-aided-manufacturing (CAM) can then be used to facilitate fabrication of the 3-D structure using SFF as described herein. Alternatively, the methods and apparatus disclosed herein may be used to produce a non-specific 3-D structure (e.g., a block or cube), which is then cut or molded into the desired shape (e.g., using a laser, saw, blade, etc.).

Rapid prototyping (RP) is a technique that can be divided into the additive and subtractive method. Additive rapid prototyping (ARP) can have the ability to create complex shapes and hollow structures. ARP is a broad category that includes many different methods including stereolithography, fused deposition modeling, direct metal laser sintering, laminated object manufacturing, electron beam melting, selective laser sintering, laser engineered net shaping, and 3-dimensional printing (3DP). 3DP does not require heat for its functionality which makes it useful for cell or growth factor incorporation. This feature made 3DP an attractive method for tissue engineering. Robocasting or direct ink writing (DIW) is a subcategory of 3DP that is based on a computer aided fabrication method that uses extrusion of the “ink” while moving in all three axes to make a 2D layer. By adding these 2D layers on top of each other, a 3D object can be created. The robocaster allows precise control of micro patterning by determining the dimensions of filaments, the size and shape of pores and the percentage of porosity of the scaffold.

In some embodiments, a composition can be made as disclosed herein and applied through a 3D printer. In one aspect, a system for printing a porous scaffold in vivo in situ is disclosed. In some embodiments, the method can comprise preparing a slurry comprising biosilicate nanoparticles and osteoinductive biopolymers; and applying the slurry, through a printer head attached to a motorized manifold; and being driven by a CAD program, to a bone fracture site.

In other embodiments, an optimized scaffold slurry can be selected; printer parameters can be calibrated; a scaffolding structure can be programmed into a CAD program; the printer head can be engaged; and the scaffold can be printed.

In other embodiments, as part of the method of some embodiments of the 3-D printing method, a composition in its final formulation can be fed into a delivery device, such as the nozzle of an automated 3-D printing device and provided as a series of layers. In another embodiment, the method may include the step of first preparing a quantity of a composition for making a scaffold. In some embodiments, the composition can comprise methacrylated chitosan and PEGDA. The composition can comprise a suitable viscosity that will permit it to be extruded through a nozzle having a extrusion component, such as a needle, the needle or other extrusion component having a size of between about a 14 to about a 32 gauge size (in some embodiments, a gauge 30 (0.2 mm) dispenser tip). Printing (i.e., delivery) of the composition may be accomplished by providing a defined quantity of a composition extruded through a small gauge dispenser tip, such as a needle having a size of about 14 (1.55 mm) to about 32 (about 0.1 mm) gauge. The method may provide any number of different configurations and geometries of a composition deposition at a desired site, such as to provide a single layer, multiple stacked or unstacked layers, mesh configuration, triangular configuration, rectangular configuration, or other configuration as may be best suited

### Biodegradable

In some embodiments, a scaffold or part thereof can be biodegradable or bioerodible. The speed of erosion of a scaffold produced from a bioerodible or biodegradable composition can be related to the molecular weights of a polymer or ingredient used in the composition. For example, higher molecular weight polymers (e.g., with average molecular weights of 90,000 or higher) produce scaffolds which may retain their structural integrity for longer periods of time, while lower molecular weight polymers (e.g., average molecular weights of 30,000 or less) may produce scaffolds which erode much more quickly.

As used herein, the term “degradable” refers to a material having a structure which can decompose to smaller molecules under certain conditions, such as temperature, abrasion, pH, ionic strength, electrical voltage, current effects, radiation and biological means. After degraded at least partially a scaffold, one or more species of living cells can be added. The method may further include incubating the scaffold under conditions which allow proliferation of the one or more species of living cells added after degrading at least partially the scaffold.

### Enhanced Scaffold

In some embodiments, a composition for use in making a scaffold can comprise elements capable of modifying, preserving or enhancing one or more characteristics of the scaffold, including, ionic concentration; pH; speed and/or extent of cross-linking of a structure or ingredient; speed and/or extent of setting or solidification of a structural a structure or ingredient; speed and/or extent of degradation; porosity; rigidity; surface adhesion properties; modification of bioavailability, residence time and/or mass transport of a structure or ingredient; and other characteristics of the 3-D biomimetic structure.

In some embodiments, a composition for use in making a scaffold can comprise elements for improving surface adhesion of the scaffold include nonfibrillar collagen, fibrillar collagen, mixtures of nonfibrillar and fibrillar collagen, methyl alpha-cyanoacrylate, methacrylate, 2-cyano-2-propenoic acid methyl ester, methyl 2-cyanoacrylate, 2-cyanoacrylic acid methyl ester, an n-butyl cyanoacrylate based glue, fibronectins, ICAMs, E-cadherins, and antibodies that specifically bind a cell surface protein (for example, an integrin, ICAM, selectin, or E-cadherin), peptides containing “RGD” integrin binding sequence, or variations thereof known to affect cellular attachment, or other biologically active cell attachment mediators.

In some embodiments, a composition for use in making a scaffold can comprise elements for producing a biological effect (e.g., stimulation or suppression of cell division, migration or apoptosis; stimulation or suppression of an immune response; anti-bacterial activity; etc.). Therapeutic bio-inks may comprise one or more agents, as described more fully below, in a single ink. In some embodiment, such elements can comprise osteoinductive, angiogenic, mitogenic, or similar substances, such as transforming growth factors (TGFs), for example, TGF-alpha, TGF-beta-1, TGF-beta-2, TGF-beta-3; fibroblast growth factors (FGFs), for example, acidic and basic fibroblast growth factors (aFGF and bFGF); platelet derived growth factors (PDGFs); platelet-derived endothelial cell growth factor (PD-ECGF); tumor necrosis factor alpha (TNF-alpha); tumor necrosis factor beta (TNF-b); epidermal growth factors (EGFs); connective tissue activated peptides (CTAPs); osteogenic factors, for example, for example, BMP-1, BMP-2, BMP-3MP-4, BMP-5, BMP-6, BMP-7, BMP-8, BMP-9; insulin-like growth factor (IGF), for example, IGF-I and IGF-II; erythropoietin; heparin binding growth factor (hbgf); vascular endothelium growth factor (VEGF); hepatocyte growth factor (HGF); colony stimulating factor (CSF); macrophage-CSF (M-CSF); granulocyte/macrophage CSF (GM-CSF); nitric oxide synthase (NOS); nerve growth factor (NGF); muscle morphogenic factor (MMP); Inhibins (for example, Inhibin A, Inhibin B); growth differentiating factors (for example, GDF-1); Activins (for example, Activin A, Activin B, Activin AB); angiogenin; angiotensin; angiopoietin; angiotropin; antiangiogenic antithrombin (aaAT); atrial natriuretic factor (ANF); betacellulin; endostatin; endothelial cell-derived growth factor (ECDGF); endothelial cell growth factor (ECGF); endothelial cell growth inhibitor; endothelial monocyte activating polypeptide (EMAP); endothelial cell-viability maintaining factor; endothelin (ET); endothelioma derived mobility factor (EDMF); heart derived inhibitor of vascular cell proliferation; hematopoietic growth factors; erythropoietin (Epo); interferon (IFN); interleukins (IL); oncostatin M; placental growth factor (PIGF); somatostatin; transferring; thrombospondin; vasoactive intestinal peptide; and biologically active analogs, fragments, and derivatives of such growth factors. In some embodiments, the elements may comprise polynucleotides. Examples of polynucleotides include, but are not limited to, nucleic acids and fragments of nucleic acids, including, for example, DNA, RNA, cDNA and recombinant nucleic acids; naked DNA, cDNA, and RNA; genomic DNA, cDNA or RNA; oligonucleotides; aptomeric oligonucleotides; ribozymes; anti-sense oligonucleotides (including RNA or DNA); DNA coding for an anti-sense RNA; DNA coding for tRNA or rRNA molecules (i.e., to replace defective or deficient endogenous molecules); double stranded small interfering RNAs (siRNAs); polynucleotide peptide bonded oligos (PNAs); circular or linear RNA; circular single-stranded DNA; self-replicating RNAs; mRNA transcripts; catalytic RNAs, including, for example, hammerheads, hairpins, hepatitis delta virus, and group I introns which may specifically target and/or cleave specific RNA sequences in vivo; polynucleotides coding for therapeutic proteins or polypeptides, as further defined herein; chimeric nucleic acids, including, for example, DNA/DNA hybrids, RNA/RNA hybrids, DNA/RNA hybrids, DNA/peptide hybrids, and RNA/peptide hybrids; DNA compacting agents; and gene/vector systems (i.e., any vehicle that allows for the uptake and expression of nucleic acids), including nucleic acids in a non-infectious vector (i.e., a plasmid) and nucleic acids in a viral vector. In an exemplary embodiment, chimeric nucleic acids, include, for example, nucleic acids attached to a peptide targeting sequences that directs the location of the chimeric molecule to a location within a body, within a cell, or across a cellular membrane (i.e., a membrane translocating sequence (“MTS”)). In another embodiment, a nucleic acid may be fused to a constitutive housekeeping gene, or a fragment thereof, which is expressed in a wide variety of cell types.

The term “cross-linking agent” can refer to an agent which induces cross-linking. The cross-linking agent can be any agent that is capable of inducing a chemical bond between adjacent polymeric chains. For example, the cross-linking agent can be a chemical compound. Examples of chemical compounds that can act as cross-linking agent include, but are not limited to, 1-ethyl-3[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methymethacrylate, N,N′-methylene-bisacrylamide, N,N′-methylene-bis-methacrylamide, diallyltartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase or mixtures thereof.

### Leather Articles

A synthetic leather herein can be at least a portion of a leather article. For example, a synthetic leather can be used as substitute of natural leather in a leather article. Exemplary leather articles include a watch strap, belt, suspender, packaging, shoe, boot, footwear, glove, clothing (e.g., tops, bottoms, and outerwear), luggage, bag (e.g., a handbag with or without shoulder strap), clutch, purse, coin purse, billfold, key pouche, credit card case, pen case, backpack, cases, wallet, saddle, harness, whip, travel goods (e.g., a trunk, suitcase, travel bag, beauty case, or a toilet kit), rucksacks, portfolio, document bag, briefcase, attache case, pet article (e.g., a leash or collar), hunting and fishing article (e.g., a gun case, cutlery case, or a holster for firm arms), a stationary article (e.g., a writing pad, book cover, camera case, spectacle case, cigarette case, cigar case, jewel case, or a mobile phone holster), or a sport article (e.g., a ball such as basketball, soccer ball, or a football). For example, a leather article can be a watch wrap. For example, a leather article can be a belt. For example, a leather article can be a bag.

### Example 1. Exemplary Scalable, Modular Cell Culture System

A leather production module was constructed with the following components:
(1) 1× Caron 7400-33 Reach-In Carbon Dioxide (CO2)/Relative Humidity (RH) Incubator
(2) 1× Ultramotion 2-B.125-STM652-8-1NO-EC4/EC4-ESB Linear Actuator (8″ Stroke Length)
3× Cole-Parmer 07575-50 MasterFlex L/S Pumps with EtherNet/IP
   (3) Stock Pump
   (4) Feed Pump
   (5) Waste Pump
11× Cole-Parmer 77202-60 MasterFlex L/S Easy-Load-II Pump Heads (2-Channels/Head)
   (6) 1× Pump Head on Stock Pump
   (7) 5× Pump Heads on Feed Pump
   (8) 5× Pump Heads on Waste Pump
   (9) 1× VitroLabs'Custom-Designed Rack and Shelf System (Fabricated by Ideal Prototypes)
   (10) 1× VitroLabs'Custom-Designed Automation System (Assembled and Programmed by Calcon Systems)
Various Lengths of C-Flex 374 tubing (e.g., ⅛″ ID×¼″ OD Saint Gobain 374-125-2)
   (11) Tubing from Bioprocess bag in refrigerator (i.e., the cold media storage bag) to Bioprocess bag on top shelf of the incubator (i.e., the media warming bag)
   (12) Tubing from Bioprocess bag on top shelf of the incubator (i.e., the media warming bag) to the 10 individual containers
   (13) Tubing from 10 individual containers to Bioprocess bag on bottom shelf of Metro rack (i.e., the waste bag)
(14) 1× Refrigerator with side port (K2Scientific K210SDF)
(15) 10× Dynalon Polypropylene 15″×20″×3″ Rectangular Trays (107314) and associated 15.5″×20.5″×1″ Lids (L731) (i.e., the Rectangular Containers). The shelves are designed to accommodate 20″×20″×3″ Rectangular Trays (to be custom fabricated by Dynalon). Alternatively, we have utilized 10× Origen Biomedical EV3000N bags (gas permeable).
(16) 10× VitroLabs'Custom-Designed 22 cm×8 cm stainless steel wire cloth frames (laser cut by BT Laser from McMaster-Carr 9319T141 316 Stainless Steel Wire Cloth, 10×10 mesh, 0.065″ Opening)
(17) 10× Cell Culture Substrates (e.g., a 100% recycled polyethylene terephthalate (PET) polyester batting (Evergreen™, 3.5 oz./sq. yd, nominally 2.5 mm thick; Fairfield Processing Corp., Danbury, CT).
3× Bioprocess bags (e.g., 20 L Labtainer™ BioProcess container; Thermo Fisher)
   (18) Bioprocess bag in refrigerator (i.e., the cold media storage bag)
   (19) Bioprocess bag on top shelf of the incubator (i.e., the media warming bag)
   (20) Bioprocess bag on bottom shelf of Metro rack (i.e., the waste bag)
   (21) 1× Metro rack (or equivalent)

### Example 2: Production of Cell Culture in a Modular, Scalable Bioreactor

A representative bioreactor process begins by preparing cell culture substrates (FIG. 2; 17). Sheets of 100% recycled PET batting (Fairfield) are cut to size (e.g., 22 cm×8 cm, for an EV3000N bioreactor bag) and affixed to the 316 stainless steel wire cloth frames by way of stainless-steel wire (FIG. 2; 16). The cell culture substrates are then either sealed in breathable sterilization pouches and autoclave sterilized or placed directly into polypropylene containers (FIG. 2; 15) and autoclave sterilized in situ. In an alternative process, autoclave sterilized cell culture substrates are aseptically introduced into pre-sterilized EV3000N gas-permeable bioreactor bags, which are then heat sealed to reestablish a sterile boundary. The sterilized individual polypropylene containers or gas-permeable bioreactor bags (FIG. 2; 15) are then positioned on the shelves of the custom-designed rack and shelf system (FIG. 2; 9). Of note, during assembly of the Prototype Production Module, communication cables (e.g., Ethernet; not shown) from the programmable logic controller (PLC) of the custom-designed automation system (FIG. 2; 10) are routed to the stock pump (FIG. 2; 3), feed pump (FIG. 2; 4), and waste pump (FIG. 2; 5) as well as to the linear actuator (FIG. 2; 2). The linear actuator (FIG. 2; 2) is mechanically linked to the custom-designed rack and shelf system (FIG. 2; 9) in such a way that extension and retraction of the linear actuator is capable of enabling tilting and/or a rocking motion of the shelves.

The bioreactor process further involves connecting the media warming bag (FIG. 2; 19) to the cold media storage bag (FIG. 2; 18) in the refrigerator (FIG. 2; 14) via tubing (FIG. 2; 11). This tubing (FIG. 2; 11) is positioned in the pump head (v 2; 6) of the stock pump (FIG. 2; 3). The 10× individual containers (FIG. 2; 15) are connected to the media warming bag (FIG. 2; 19) via tubing (FIG. 2; 12). This tubing (FIG. 2; 12) is positioned in the 5× pump heads (FIG. 2; 7) of the feed pump (FIG. 2; 4). The 10× individual containers (FIG. 2.; 15) are connected to the waste bag (FIG. 2; 20) on the Metro rack (FIG. 2; 21) via tubing (FIG. 2; 13). This tubing (FIG. 2; 13) is positioned in the 5× pump heads (FIG. 2; 8) of the waste pump (FIG. 2; 5).

At this stage, all of the containers and bags are connected via tubing that is positioned in the pump heads of the pumps, thus completing the setup of the bioreactor sterile boundary. If not already running, the Caron incubator (FIG. 2; 1) is turned on at this time and the temperature is set (e.g., 37C). The water pan of the Caron incubator is filled with sterilized water to provide relative humidity. Of note, the cold media storage bag can be filled via routine bioprocessing techniques ahead of connecting to the system or can be filled at this time. Also, in some representative processes, cell seeding of the cell culture substrates (FIG. 2; 17) can be performed ahead of connecting the individual containers (e.g., by aseptically pipetting a suspension of cells in culture media onto the cell culture substrates inside the individual containers beneath a biological safety cabinet). In some alternative processes, cell seeding of the cell culture substrates (FIG. 2; 17) can be performed after connecting the 10× individual containers (FIG. 2; 15) (e.g., by temporarily connecting a bag of cell suspension to the 10× individual containers in place of the media warming bag and then utilizing the feed pump (FIG. 2; 4) to introduce cell suspension into the 10× individual containers for a prescribed period of time prior to introducing warmed media).

Subsequent steps of the process are administrated via the custom-designed automation system (FIG. 2; 10). The automated aspects of a representative bioreactor process are initiated by first homing the linear actuator via a jog button on the human-machine interface (HMI) of the automation system (FIG. 2; 10) and magnetic limit switch on the linear actuator (FIG. 2; 2). In a representative process, the home position refers to the retracted position of the linear actuator. Via the HMI of the automation system (FIG. 2; 10), an operator is capable of entering and uploading a variety of process parameters related to the pumps and sequence of pumping operations, including, for example, the tubing size, flow rate, and dispense volume as well as the time delay between pump cycles. Via the HMI, an operator is also capable of entering and uploading a variety of process parameters related to the linear actuator and sequence of linear actuator operations, including, for example, the current to provide to the motor of the actuator, the rocking speed and the rocking angle. In a representative process involving 10×EV3000N bioreactor bag as the individual containers, each containing a 22 cm×8 cm cell culture substrate, a representative subset of stock pump parameters includes a tubing size of L/S 16, a flow rate of 100 mL/min, and a dispense volume of 1250 mL. A representative subset of feed and waste pump process parameters include a tubing size of L/S 16, a flow rate of 100 mL/min, and a dispense volume of 125 mL, and a time delay between pump cycles of 48 hours. A representative subset of linear actuator parameters includes a linear speed of 2.5 inches per second and a linear displacement corresponding with a rocking angle of 5 to 30 degrees.

Once the process parameters described above are entered and uploaded and the program is initiated, the custom-designed automation system (FIG. 2; 10) controls the pumps and actuator in a repeating sequence, in which, for representative process parameters indicated above: (a) the linear actuator will rock the shelves back and forth about their central axis at the prescribed speed and angle for 48 hours; (b) at 46 hours from initiating the program, the stock pump (FIG. 2; 3) will dispense 1250 L from the cold media storage bag (FIG. 2; 18) to the media warming bag (FIG. 2; 19) (during this time, the linear actuator continues rocking the shelves); (c) at 48 hours, the linear actuator retracts, thereby positioning the shelves (and individual containers positioned on the shelves) at an angle to enable waste removal; (d) the waste pump (FIG. 2; 5) removes 125 mL of media from each of the 10× individual containers (FIG. 2; 15) into the waste bag (FIG. 2; 20); (e) the feed pump (FIG. 2; 4) adds 125 mL of warm media from the media warming bag (FIG. 2; 19) into each of the 10× individual containers (FIG. 2; 15). At this point, the waste removal and feeding cycle is complete and the linear actuator reinitiates rocking the shelves and the 48-hour cycle begins again and repeats every 48 hours for the duration of the bioreactor process. At prescribed intervals (e.g., every 1 week), the cold media storage bag (FIG. 2; 18) is refilled with freshly filtered media, utilizing standard bioprocess techniques, and the full waste bag is likewise replaced with a new, empty waste bag.

### Example 3: Preparation of Chitosan Resin

Chitosan powder (fungal source, low MW 50,000-190,000 DA>75%) can be mixed with 3% (w/v) glacial acetic acid by stirring overnight at 60 degrees C until fully dissolved. Methacrylic anhydride can then be added in excess (12 ml per 500 ml chitosan solution) at 60C, well shaken, and left to stir overnight. Once methacrylation has started, the solution should be kept out of light.

The solution can be dialysed with a MWCO of 3500 Da in 3% acetic acid for a minimum of 5 dilutions. In this case using 4 L per dilution. Each dilution can be a minimum of 3 hours. The chitosan solution can then be lyophilized for a minimum of 36 hours. Once fully dried, the chitosan can be resuspended overnight in a 3% acetic acid mixture in dPBS, or other cell culture media that is phenol red free.

### Example 4: Preparation of Pegda Resin

For a final volume of 1 L of resin, PEGDA (700 mw)-3-6, meaning the resin contains 0.3% Li-TPO, and 0.06% Martius Yellow can be made. For 500 ml of PEGDA, 3 grams of finely ground Li-TPO can be added to 40 ml of solvent, in this case dPBS. The mixture can be stirred at 60 degrees C until fully dissolved. From this point on, the solution should be kept out of light. Once dissolved, the solvent can be added to 460 ml of PEGDA-700 and return to stirring at 60 degrees C. 600 mg of Martius Yellow can be added to the solution. Observation suggests that resin reaches ideal state after 3-4 days of mixing, though it will function before this in some diminished capacity.

### Example 5: Combining Methacrylated Chitosan With Pegda Resin

Methacrylated chitosan of example 3 can be mixed with PEGDA resin of example 4 can be mixed together 1:1 and left to stir at 60 degrees C overnight (protected from light), or longer depending on how long the PEGDA has been stirring for.

### Example 6: Scaffold Print Files

Basic geometry can be generated using Autodesk Meshmixer, in this case squares or rectangles with a thickness of 2 mm. This geometry can then be exported as an STL and then converted into a scaffold using Autodesk within Medical. The shape can be imported, and a trabecular lattice can be generated with beam size 0.1 mm pore size 0.2 mm, and advanced edge cleaning. The lattice can then be exported as an STL back to meshmixer to add a frame around the lattice, typically 0.5 mm thick, and 0.5 mm taller than the lattice. This shape can be imported as an STL into Autodesk Netfabb. Netfabb can be used to slice the shape into 50 um layers, then cleaned up by removing all self-intersections. The file can then be converted to a format usable by the Ember printer and also able to take advantage of the high-resolution pattern mode. This can entail rotating the layers 45 degrees counterclockwise and then exporting the layers as a zip file of PNG images, with a DPI of 508, and image size of 1482×1482.

The PNG files can then have their names changed from “layer_0x” to “slice_x” so that the Ember could read the zip file. This can be accomplished using a custom python script. The next steps can be the creation of a print settings file containing exposure times (typically 1-2 seconds per layer) motor speeds, and the modification of the first 5-8 layers of the print file to be identical. This can be done to minimize the likelihood of the first few layers of the print becoming a solid mass.

While some embodiments have been shown and described herein, such embodiments are provided by way of example only. Numerous variations, changes, and substitutions will now occur to those skilled in the art without departing from the disclosure provided herein. It should be understood that various alternatives to the embodiments described herein can be employed.

### Example 7: Manufacturing Process

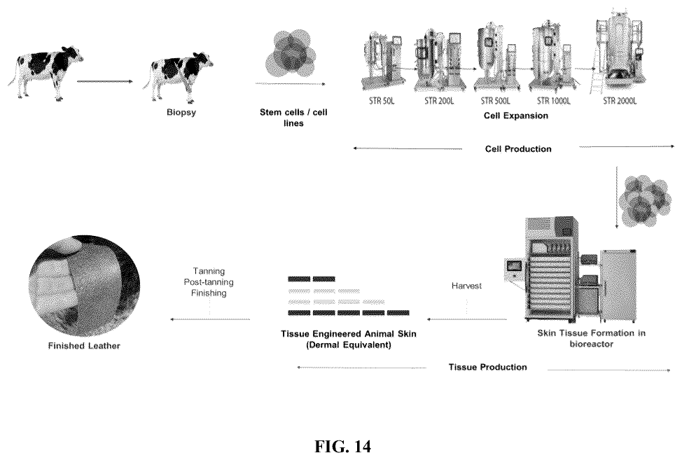

Cells used for manufacturing of product were fetal bovine fibroblasts sourced initially from a biopsy of a cow. Cells were plated and expanded on 2D plasticware. A serum-containing media was used for cell expansion. Cells were seeded onto needle-punched, non-woven recycled polyester. Cells were contacted with tissue formation media, which consisted of basal media with 10% human platelet lysate, ascorbate and TGFβ. Tissues were cultured under static conditions for one week @ 37° C./5% CO₂ and then placed on a rocker to generate a wave form across the surface of the material and mix the media to provide gas exchange. Tissues were generally cultured between 6.5 and 9.5 weeks. Harvested “skins” were generally immersed in either a 36% salt brine or immersed in salt crystals to preserve the tissue. Preserved skins were tanned and dyed. An outline of the process is depicted in FIG. 14.

### Example 8: Tissue Bioreactor

A tissue bioreactor uses a continuous wave motion of fluid across cells on a biomaterial surface to generate shear. Shear across the surface can enhance collagen production and support nutrient and gas exchange to cells. A pallet system can support growth of approximately 26 layers of 3 ft×3 ft sheets of tissue.

### Example 9: Large Scale Hide Reactor (LSHR)

Tissues can be grown in either a horizontal or vertical position. The initial design is targeted for a 5 ft×3.5 ft sheet of tissue. Multiple layers will be grown in the same bioreactor. Multiple bioreactors (cassettes) can be connected to the same reservoir. Multiple cassettes can be placed in a climate-controlled box. Baffles are used to direct media flow past each layer. Cells are seeded onto scaffolds either inside a bioreactor or sprayed onto scaffolds prior to loading into a bioreactor. Bioreactor components will be designed to be reused several times to reduce environmental impact. Bioreactor may be designed to be cleaned and sterilized in place. A reservoir of media will be connected to a bioreactor to provide extra media volume to a system. Media will be recycled from a reservoir to the bioreactor and back. Sensors for pH, oxygen, and glucose will be placed in the reservoir to control when media needs to be removed, and new media needs to be added.

What is claimed is:

1. A tissue culturing device configured for production of a synthetic leather, the tissue culturing device comprising at least one cell culturing container, wherein the at least one cell culturing container comprises a scaffold, wherein the at least one cell culturing container comprises a fibroblast cell or a fibroblast expressing CD10, CD73, CD44, CD90, type 1 collagen, type III collagen, or any combination thereof, wherein the tissue culturing device is a fixed bed perfusion bioreactor.

2. The tissue culturing device of claim 1, wherein the tissue culturing device is temperature controlled.

3. The tissue culturing device of claim 1 or 2, wherein the tissue culturing device is jacketed.

4. The tissue culturing device of anyone of claims 1 to 3, wherein the tissue culturing device comprises a monitoring system.

5. The tissue culturing device of claim 4, wherein the monitoring system comprises a sensor, a camera, or a combination thereof.

6. The tissue culturing device of claim 5, wherein the monitoring system comprises the sensor, wherein the sensor comprises a thermistor, a thermometer, a pH sensor, a humidity sensor, a pressure sensor, a gas sensor, a CO₂ sensor, an O₂ sensor, a smoke detector, or any combination thereof.

7. The tissue culturing device of any one of_claims 1 to 6, wherein the fibroblast cell or the fibroblast expressing CD10, CD73, CD44, CD90, type 1 collagen, type III collagen, or any combination thereof, comprises a plurality of fibroblast cells expressing CD10, CD73, CD44, CD90, type 1 collagen, type III collagen, or any combination thereof, in a cell layer.

8. The tissue culturing device of claim 7, wherein the cell layer is in contact with the scaffold.

9. The tissue culturing device of any one of claims 1 to 8, wherein the tissue culturing device comprises an agitator configured to mix growth medium within a container.

10. The tissue culturing device of any one of claims 1 to 9, wherein the tissue culturing device comprises at least one, two, three, four, five, six, seven, eight, ten, or fifteen openings or ports.

11. A system comprising the tissue culturing device of any one of claims 1 to 10 and a cell culture medium reservoir.

12. The system of claim 11, wherein the cell culture medium reservoir is maintained at a lower temperature than the tissue culturing device.

13. The system of claims 11 or 12, wherein the cell culture medium is warmed before entering the

14. The system of any one of claims 11 to 13, wherein the tissue culturing device comprises an outlet for waste cell culture medium.
